SEQUENCE LISTING (101 sequences)

SEQ ID NO: 1 - protein, 9 aa, Homo sapiens
FIARNYLEG

SEQ ID NO: 2 - protein, 16 aa, Homo sapiens
FIARNYLEDPEDERRK

SEQ ID NO: 3 - protein, 7 aa, Homo sapiens
LEPAILE

SEQ ID NO: 4 - protein, 52 aa, Homo sapiens
LEPGKECVQPATKSCSFSACTDGISWPFVLLIMPLVIWVYSTDTNFSDMFWS

SEQ ID NO: 5 - protein, 10 aa, Homo sapiens
HHVDEKKDLK

SEQ ID NO: 6 - protein, 12 aa, Homo sapiens
CDPKLSTTDRVV

SEQ ID NO: 7 - protein, 25 aa, Homo sapiens
VQQDGFNSLNTAHTTENHGTGNHSA

SEQ ID NO: 8 - protein, 18 aa, Homo sapiens
KWQIVHFHRSGAPSVLPH

SEQ ID NO: 9 - protein, 15 aa, Homo sapiens
RRGSGTPEAEAPRQW

SEQ ID NO: 10 - protein, 16 aa, Homo sapiens
QNVHFHRSGSPTVPIK

SEQ ID NO: 11 - protein, 15 aa, Homo sapiens
RSPEGRSSRDRTAPS

SEQ ID NO: 12 - protein, 14 aa, Rattus rattus
CTRFTDEYQLFEEL

SEQ ID NO: 13 - protein, 14 aa, Rattus rattus
  MISC_FEATURE (6)..(6): Phosphothreonine
MHRQETVDCLKKFN

SEQ ID NO: 14 - protein, 11 aa, Homo sapiens
EGEKVRVGDDL

SEQ ID NO: 15 - protein, 11 aa, Homo sapiens
FLRFAVFCNGE

SEQ ID NO: 16 - protein, 11 aa, Homo sapiens
WDAVVGFLHVF

SEQ ID NO: 17 - protein, 11 aa, Homo sapiens
FVKRKVMDKYG

SEQ ID NO: 18 - protein, 11 aa, Homo sapiens
EGEKVQVGDDL

SEQ ID NO: 19 - protein, 11 aa, Homo sapiens
FLRFAIFCNGE

SEQ ID NO: 20 - protein, 11 aa, Homo sapiens
WDAVVSFLHVF

SEQ ID NO: 21 - protein, 11 aa, Homo sapiens
FVKRKFMDKYG

SEQ ID NO: 22 - protein, 11 aa, Homo sapiens
GGMNRRPILTI

SEQ ID NO: 23 - protein, 11 aa, Mus musculus
TEVVRRCPHHE

SEQ ID NO: 24 - protein, 11 aa, Mus musculus
TEVVRRCPHHE

SEQ ID NO: 25 - protein, 11 aa, Mus musculus
DSFEVRVCACP

SEQ ID NO: 26 - protein, 11 aa, Homo sapiens
GGMNRSPILTI

SEQ ID NO: 27 - protein, 11 aa, Mus musculus
TEVVRPCPHHE

SEQ ID NO: 28 - protein, 11 aa, Mus musculus
TEVVRHCPHHE

SEQ ID NO: 29 - protein, 11 aa, Mus musculus
DSFEVHVCACP

SEQ ID NO: 30 - protein, 16 aa, Homo sapiens
KYHGVSLLNPPETLNL

SEQ ID NO: 31 - protein, 11 aa, Homo sapiens
CTPTDVRDVDI

SEQ ID NO: 32 - protein, 20 aa, Homo sapiens
MATSRYEPVAEIGVGAYGTV

SEQ ID NO: 33 - protein, 11 aa, Homo sapiens
CSQNTSELNTA

SEQ ID NO: 34 - protein, 8 aa, Mus musculus
LGTPNNEV

SEQ ID NO: 35 - protein, 149 aa, Homo sapiens
SMEPSADWLATAAARGRVEEVRALLEAGALPNAPNSYGRRPIQVMMMGSARVAELLLLHGAEPNCADPATLTRPVHDAAREGFLDTLVVLHRAGARLDVRDAWGRLPVDLAEELGHRDVARYLRAAAGGTRGSNHARIDAAEGPSDIPD

SEQ ID NO: 36 - protein, 11 aa, Homo sapiens
CTPTDVRDVDI

SEQ ID NO: 37 - protein, 59 aa, Homo sapiens
SPPPSSPPSSLTTDPSQSLLSLEQEPLLSRMGSLRAPVDEDRLSPLVAADSLLEHVRED

SEQ ID NO: 38 - protein, 17 aa, Homo sapiens
CKYHGVSLLNPPETLNL

SEQ ID NO: 39 - protein, 198 aa, Homo sapiens
MSNVRVSNGSPSLERMDARQAEHPKPSACRNLFGPVDHEELTRDLEKHCRDMEEASQRKWNFDFQNHKPLEGKYEWQEVEKGSLPEFYYRPPRPPKGACKVPAQESQDVSGSRPAAPLIGAPANSEDTHLVDPKTDPSDSQTGLAEQCAGIRKRPATDDSSTQNKRANRTEENVSDGSPNAGSVEQTPKKPGLRRRQT

SEQ ID NO: 40 - protein, 66 aa, Homo sapiens
MESHSVTQAGVQWRDLGSLQPLPPGFKQFSHLSLPSSWDYRRVPPYLGNFCIFSGEGVSPCWPGWS

SEQ ID NO: 41 - protein, 12 aa, Homo sapiens
EEVDLQDLPSAT

SEQ ID NO: 42 - protein, 248 aa, Homo sapiens
QDLSEGISQKDGDEVCESNVSNSITSGEKVISVELGLDNGHSAIQTGTLILEEIETIKEKQDVQPQQASPLETSETDHQQPVLSDVPPLPAIPDQQIVEEASNSTLESAPNGKDYESTEIVAEETKPKDTELSQESDFKENGITEEKSKSEESKRMEPIAIIITDTEISEFDVTKSKNVPKQFLISAENEQVGVFANDNGFEDRTSEQYETLLIETASSLVKNAIQLSIEQLVNEMASDDNKINNLLQ

SEQ ID NO: 43 - protein, 164 aa, Homo sapiens
MVNPTVFFDITADDEPLGRVSFELFADKVPKTAENFRALSTGEKGFGYKGSSFHRIIPGFMCQGGDFTRHNGTGGRSIYGEKFEDENFILKHTGPGILSMANAGPNTNGSQFFICTAKTEWLDGKHVVFGKVKEGMNIVEAMERFGSRNGKTSKKITISDCGQL

SEQ ID NO: 44 - protein, 110 aa, Homo sapiens
MIRIAALNASSTIEDDHEGSFKSHKTQTKEAQEAEAFALYHKALDLQKHDRFEESAKAYHELLEASLLREAVSSGDEKEGLKHPGLILKYSTYKNLAQLAAQREDLETAM

SEQ ID NO: 45 - protein, 15 aa, Homo sapiens
KKGSEQESVKEFLAK

SEQ ID NO: 46 - protein, 15 aa, Homo sapiens
CAHSFFDELRDPNVK

SEQ ID NO: 47 - protein, 195 aa, Homo sapiens
HIDQWNKVIEQLGTPSAEFMKKLQPTVRNYVENRPKYPGIKFEELFPDWIFPSESERDKIKTSQARDLLSKMLVIDPDKRISVDEALRHPYITVWYDPAEAEAPPPQIYDAQLEEREHAIEEWKELIYKEVMDWEERSKNGVVKDQPSDAAVSSNATPSQSSSINDISSMSTEQTLASDTDSSLDASTGPLEGCR

SEQ ID NO: 48 - protein, 20 aa, Homo sapiens
TYDEVISFVPPPLDQEEMES

SEQ ID NO: 49 - protein, 41 aa, Homo sapiens
QAGRLGSTIFVANLDFKVGWKKLKEVFSIAGTVKRADIKED

SEQ ID NO: 50 - protein, 19 aa, Mus musculus
AISSPSGLAYPDDVLDYGL

SEQ ID NO: 51 - protein, 196 aa, Homo sapiens
VIRDLLHSAIWLDVRKLAFSVPEKIQLLRIMSSSFAKKISLKQCLFTDATVPEIVSILEGMPKLMEVEMKDCTLLGTGKWYKQIHANQSQSLRILTIENLSIEEFYLFTDLQSVLDLLSLFRKVTVENTKVFLVPCKLSQHLLSLEYLDLSANLLGDQSLEHSACQGAWPSLQTLNLSQNSLSDLKMTGKSLFHLR

SEQ ID NO: 52 - protein, 18 aa, Mus musculus
ERGLSGLFRNLTLERVVE

SEQ ID NO: 53 - protein, 14 aa, Homo sapiens
DQSEAPVRQNHEEI

SEQ ID NO: 54 - protein, 195 aa, Homo sapiens; identical to SEQ ID NO: 47

SEQ ID NO: 55 - protein, 50 aa, Homo sapiens
ALLSLRLGSDAHGTELPSPPSVWFEAEFFHHILHWTPIPNQSESTCYEVA

SEQ ID NO: 56 - protein, 67 aa, Homo sapiens
SPYSSDTTPCCFAYIARPLPRAHIKEYFYTSGKCSNPAVVFVTRKNRQVCANPEKKWVREYINSLEM

SEQ ID NO: 57 - protein, 28 aa, Homo sapiens
CVETFGTTSYYDDVGLLCEKADTRALMA

SEQ ID NO: 58 - protein, 17 aa, Homo sapiens
EEFATEGTDRKDVFFYC

SEQ ID NO: 59 - protein, 15 aa, Homo sapiens
CHYPIVPLDGKGTLL

SEQ ID NO: 60 - protein, 13 aa, Homo sapiens
VQCKDKEATKLTE

SEQ ID NO: 61 - protein, 84 aa, Homo sapiens
SAKSVFSSKALVKMQLLKDVVGNDTYRINNKYDETYPPLPVEEIIKRSEFVIGQEVAYNLLVNNCEHFVTLLRYGEGVSEQANR

SEQ ID NO: 62 - protein, 16 aa, Homo sapiens
KDKKLFLKRDNLLIAD

SEQ ID NO: 63 - protein, 13 aa, Homo sapiens
KAKAKPVTRGAGA

SEQ ID NO: 64 - protein, 12 aa, Homo sapiens
GAPDYENLQELN

SEQ ID NO: 65 - protein, 13 aa, Homo sapiens
SISQVNHKPLRTA

SEQ ID NO: 66 - protein, 335 aa, Homo sapiens
TLTCMQHYEEVQTSASDLLDSQDSTGKPKCHQSRELPRIPPESAVDTMLTARSVDGDQGLGMEGPYEVLKDSSSQENMVEDCLYETVKEIKEVAAAAHLEKGHSGKAKSTSASKELPGPQTEGKAEFAEYASVDRNKKCRQSVNVESILGNSCDPEEEAPPPVPVKLLDENENLQEKEGGEAEESATDTTSETNKRFSSLSYKSREEDPTLTEEEISAMYSSVNKPGQLVNKSGQSLTVPESTYTSIQGDPQRSPSSCNDLYATVKDFEKTPNSTLPPAGRPSEEPEPDYEAIQTLNREEEKATLGTNGHHGLVPKENDYESISDLQQGRDITRL

SEQ ID NO: 67 - protein, 70 aa, Homo sapiens
DLGNSRFLDPRNKISFKFDHLKEASDPQPEEADGGLKSWRESCDSALRAYVKDHYSNGFCTVYAKTIDGQ

SEQ ID NO: 68 - protein, 14 aa, Homo sapiens
LTMSVTLSPLRSQC

SEQ ID NO: 69 - protein, 15 aa, Homo sapiens
KKGSEQESVKEFLAK

SEQ ID NO: 70 - protein, 16 aa, Homo sapiens
DHSVLTLQLPVTASVR

SEQ ID NO: 71 - protein, 24 aa, Homo sapiens
KAEEDPEAADSGEPQNKRTPDLPE

SEQ ID NO: 72 - protein, 16 aa, Mus musculus
LRAMLVYTPCHSLAER

SEQ ID NO: 73 - protein, 17 aa, Homo sapiens
AFEYEDEDGDRITVRSC

SEQ ID NO: 74 - protein, 14 aa, Homo sapiens
MAEPLKEEDGEDGS

SEQ ID NO: 75 - protein, 113 aa, Homo sapiens
IVVEWTNNTLPLEPCPKEQIAGLSRRIAEDDGKPRPAFSNALEPDFKATSITVTGSGSCRHLQFIPVVPPRRVPSEAPPTEVPDRDPEKSSEDDVYLHTVIPAVVVAAILLIA

SEQ ID NO: 76 - protein, 17 aa, Homo sapiens
MSGGKYVDSEGHLYTVP

SEQ ID NO: 77 - protein, 155 aa, Homo sapiens
ESIMGFRYRCQQCHNYQLCQDCFWRGHAGGSHSNQHQMKEYTSWKSPAKKLTNALSKSLSCASSREPLHPMFPDQPEKPLNLAHIVPPRPVTSMNDTLFSHSVPSSGSPFITRSSPPKDSEVEQNKMLARAAPAFLKGRGIQYSLNVADRLADEH

SEQ ID NO: 78 - protein, 318 aa, Homo sapiens
MAAAAAAAAEQQSSNGPVKKSMREKAVERRSVNKEHNSNFKAGYIPIDEDRLHKTGLRGRKGNLAICVIILLFILAVINLIITLVIWAVIRIGPNGCDSMEFHESGLLRFKQVSDMGVIHPLYKSTVGGRRNENLVITGNNQPIVFQQGTTKLSVENNKTSITSDIGMQFFDPRTQNILFSTDYETHEFHLPSGVKSLNVQKASTERITSNATSDLNIKVDGRAIVRGNEGVFIMGKTIEFHMGGNMELKAENSIILNGSVMVSTTRLPSSSSGDQLGSGDWVRYKLCMCADGTLFKVQVTSQNMGCQISDNPCGNTH

SEQ ID NO: 79 - protein, 6 aa, Homo sapiens
HHHHHH

SEQ ID NO: 80 - protein, 25 aa, Homo sapiens
KRRWKKNFIAVSAANRFLLISSGAL

SEQ ID NO: 81 - protein, 13 aa, Homo sapiens
DQQEQVKEDMETK

SEQ ID NO: 82 - protein, 20 aa, Homo sapiens
DIDGDGQVNYEEFVQMMTAK

SEQ ID NO: 83 - protein, 14 aa, Homo sapiens
CTRFTDEYQLFEEL

SEQ ID NO: 84 - protein, 13 aa, Homo sapiens
GVQVETISPGDGR

SEQ ID NO: 85 - protein, 18 aa, Homo sapiens
MSDSEKLNLDSIIGRLLE

SEQ ID NO: 86 - protein, 50 aa, Homo sapiens
MAYPGHPGAGGGYYPGGYGGAPGGPAFPGQTQDPLYGYFAAVAGQDGQID

SEQ ID NO: 87 - protein, 60 aa, Homo sapiens
EGLNFPTYDGKDRVVSLSEKNFKQVLKKYDLLCLYYHEPVSSDKVTQKQFQLKEIVLELV

SEQ ID NO: 88 - DNA, 696 bp, Homo sapiens
ggatccaaacgccgttggaaaaagaactttattgcagttagtgcggccaatcgctttctgctgatttcttcgggagcgctggatcagcaagaacaggttaaagaagatatggaaaccaaagatatcgatggtgatgggcaggtgaactatgaagaattcgtacagatgatgaccgccaaatgcacccgttttacggacgaataccagctgttcgaagaactgggcgttcaagttgaaacaatcagcccgggagatggccgcaaaaaaggtagtgagcaggaatctgttaaagagttcctggcgaaaatgtcagattcggagaaactgaatctggatagcattattggtcgcctgctggagatggcttacccgggtcatccgggcgcgggcggcggctattacccaggcggctatggcggtgccccgggtggtccggcgtttccgggccagacccaggatccgctgtacggctattttgcagccgtggcgggccaggatggccaaatcgatgagggcctgaatttcccgacctatgatgggaaagaccgtgttgtttcactgagcgaaaagaactttaaacaggtactgaaaaaatacgatctgctgtgcctgtattatcatgaaccagtttccagtgacaaagttacccaaaaacagtttcagctgaaggaaattgtactggaactggtgaagctt

SEQ ID NO: 89 - protein, 228 aa, Homo sapiens [sequence omitted]

SEQ ID NO: 90 - protein, 25 aa, Homo sapiens
KRRWKKNFIAVSAANRFLLISSGAL

SEQ ID NO: 91 - protein, 31 aa, Homo sapiens
ETKPNGIFKGPTAENAEYLRVAPPSSEFIGA

SEQ ID NO: 92 - protein, 23 aa, Homo sapiens
KGACHGQTGMFPRNYVTPVNRNV

SEQ ID NO: 93 - protein, 16 aa, Homo sapiens
KKSDHGNAFFPNSPSP

SEQ ID NO: 94 - protein, 13 aa, Homo sapiens
EYDPTIEDSYRKQ

SEQ ID NO: 95 - protein, 17 aa, Homo sapiens
QGPSPFTQQRDQEHFSF

SEQ ID NO: 96 - protein, 12 aa, Homo sapiens
PKKKPTPIQLNP

SEQ ID NO: 97 - protein, 13 aa, Homo sapiens
MLARRKPVLPALT

SEQ ID NO: 98 - protein, 35 aa, Homo sapiens
PFTFDMELDDLPKERLKELIFQETARFQPGAPEAP

SEQ ID NO: 99 - protein, 15 aa, Homo sapiens
VSKMLHVDPHQRLTA

SEQ ID NO: 100 - DNA, 612 bp, Homo sapiens
ggatccaaacgtcgctggaagaaaaatttcattgcggtgtcggcagcgaatcgttttctgctgatcagctctggcgctctggagacaaaaccgaacggtatctttaaagggccaacagcggaaaatgccgaatacctgcgcgtggcacctccgtcttcggagtttattggcgccaagggggcatgccacggtcagaccggtatgtttcctcgtaattatgtgacccctgttaaccgtaacgtcaaaaaatccgatcacgggaacgcattctttccgaattctccgtccccggagtacgatccgactattgaagacagttaccgtaaacagcagggacctagcccgtttacccaacagcgcgaccaggaacattttagttttccgaaaaagaaaccgacccctatccagctgaatccgatgctggctcgccgcaaaccggtcctgccagctctgaccccttttacgttcgatatggaactggatgatctgccgaaggaacgcctgaaggaactgatctttcaggaaaccgctcgttttcagccgggtgcgccggaagccccggtaagcaaaatgctgcacgtggacccgcatcagcgcctgacggcaaagctt

SEQ ID NO: 101 - protein, 200 aa, Homo sapiens
KRRWKKNFIAVSAANRFLLISSGALETKPNGIFKGPTAENAEYLRVAPPSSEFIGAKGACHGQTGMFPRNYVTPVNRNVKKSDHGNAFFPNSPSPEYDPTIEDSYRKQQGPSPFTQQRDQEHFSFPKKKPTPIQLNPMLARRKPVLPALTPFTFDMELDDLPKERLKELIFQETARFQPGAPEAPVSKMLHVDPHQRLTA

(19) United States
(12) Patent Application Publication
(10) Pub. No.: US 2009/0075392 A1
(43) Pub. Date: Mar. 19, 2009

(54) AGENTS FOR AND METHOD OF QUANITIFYING MULTIPLE RELATED COMPONENTS IN A BIOLOGICAL SYSTEM

(76) Inventor: John Colyer, Leeds (GB)

Correspondence Address: MYERS BIGEL SIBLEY & SAJOVEC, PO BOX 37428, RALEIGH, NC, 27627, US

(21) Appl. No.: 11/995,865

(22) PCT Filed: Jul. 19, 2006
(86) PCT No.: PCT/GB2006/002695

§ 371 Date: Jul. 2, 2008

(30) Foreign Application Priority Data
Jul. 19, 2005 (GB) 0514729.3
Jul. 20, 2005 (GB) 0514885.3

(51) Int. Cl. G01N 33/566; C07K 14/00; G01N 33/00

(52) U.S. Cl. 436/501; 530/350; 436/86

(57) ABSTRACT

The present invention relates to agents comprising non-natural protein sequences with at least two protein or polypeptide epitopes that are relationally linked by a common or shared functional relationship such as being components in a series of components of the same metabolic or signal transduction pathway or a pathway associated with interaction between two systems such as host-parasite or host-pathogen. The invention further includes a method of simultaneously calibrating and investigating the quantitative relationships between the at least two protein or polypeptide epitopes.

The present invention relates to agents comprising non-natural protein sequences, the sequence comprising a scaffold portion and at least two protein or polypeptide epitopes, the proteins or polypeptides being relationally linked by a functional relationship such as and without limitation, being components in a series of components of the same metabolic or signal transduction pathway or a pathway associated with interaction between two systems such as host-parasite or host-pathogen. The protein or polypeptide may also be, for example and without limitation, related by their interaction with a given protein or they may occupy a common site in a cell or they may be isoforms of the same protein or allelic variations thereof. The invention further includes a method of simultaneously calibrating and investigating the quantitative relationships between the at least two proteins or polypeptides.

### BACKGROUND

Biological science commonly involves the study of components which are in some way linked, i.e. they possess an important biological relationship with one another. The link or relationship could be that the proteins are:

1. Isoforms of one another, that is to say a protein that has the same function as another protein but is encoded by a different gene and may have small differences in its sequence. For example, transforming factor beta (TGF-B) naturally exists in three versions, or isoforms (TGF-B1, TGF-B2, and TGF-B3), each of which can set off a signalling cascade that starts in the cytoplasm and terminates in the nucleus of the cell. Alternatively isoforms can be created by alternative splicing of RNA transcribed from a gene.
2. Allelic variants of one another. Polymorphisms of a gene exist in the human population, and other species, which in some cases lead to amino acid sequence differences in the protein between individuals within the species. These polymorphisms might be without functional consequence or they might deliver benefit to the organism in particular circumstances, or they might be associated with medical disease.
3. Components of a particular process, in biology most phenomena are achieved by the coordinated action of multiple components. These are frequently called pathways, where a series of linked reactions occur within a living cell to produce a specific product or products. There are numerous metabolic pathways for example glycolysis, tricarboxylic acid or Krebs cycle, urea cycle; see Nicholson for detailed metabolic pathway maps http://www.tcd.ie/Biochemistry/IUBMB-Nicholson/), pathways in signal transduction (e.g. hormones, cytokines, growth factors, stress; several examples can be found at http://www.grt.kyushu-u.ac.ip/spad/) or pathways associated with the interaction between two systems (e.g. host-parasite interactions, host-pathogen interactions).
4. Proteins sharing a common location in the cell. The relationship could be proteins which interact with a given protein, or proteins which occupy a common site in the cell (e.g. a lipid raft).

It is known from the prior art to use biochemical assays or antibodies specific for each individual protein as herein before described to elucidate the underlying mechanisms of pathways or to detect their presence.

It is also known in the prior art to use single or multiple or his tagged proteins to detect or aid in the isolation and purifications of any of the proteins as herein before described so as to permit their quantification. However, these tagged proteins do not posses the appropriate properties which would allow them to be used for the study of components which are in some way linked and possess an important biological relationship with one another nor can they be used to study the “natural” molecule by virtue of the addition of extra protein sequence (which could influence structure or function) in the form of the epitope tag. Moreover, they do not provide any means with which to simultaneously calibrate quantitatively the amount of test product within a sample.

PCT/GB2005/00015 describes calibration complexes comprising a scaffold protein and a method of using the complexes for quantifying the amount of a target protein in a sample. A target moiety, such as a protein, is covalently attached to one or more cysteine or lysine groups on the scaffold protein and the scaffold material has a controlled property such as relative molecular mass or weight or a pH value for the isoelectric point. In this way the target moiety and scaffold protein can be used to detect not only the presence of a target protein in a sample but also as a positive control, an internal standard or may it be used to generate a calibration curve.

In the present invention we provide agents that have advantageously combined the property of a calibration standard with the ability to quantify multiple related or linked components in a biological system. In addition we describe a method using the agents of the present invention to detect not only the presence of individual relationally linked proteins or polypeptides but the quantity of each protein in a relationally linked series in a sample, in this way the method and agents of the invention may be used to provide stiochiometric information about the linked series of components in a sample.

### BRIEF SUMMARY OF THE DISCLOSURE

According to a first aspect of the invention there is provided a non-natural protein sequence, the sequence comprising a removable scaffold portion and at least two or more protein or polypeptide epitopes, the proteins or polypeptides epitopes being relationally linked.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

Reference herein to “the proteins or polypeptides being relationally linked” is intended to include a shared or common functional relationship such as being components of the same metabolic or catabolic or signal transduction pathway, or isoforms of the same protein or polypeptide, or allelic variations thereof, or proteins or polypeptides that share a common location within a cell or that interact with the same target or protein or homologue thereof.

Reference herein to an “epitope” relates to a specific chemical domain on an antigen that stimulates the production of, and is recognized by, an antibody. Each epitope on a molecule such as protein elicits the synthesis of a different antibody. aka antigenic determinant.

Reference herein to “removable scaffold” is intended to include a non-natural protein sequence which may, as an end product contain a scaffold portion or which may prior to its use have the scaffold portion removed by, for example and without limitation enzymatic means. It will be appreciated that the non-natural protein sequence of the present invention when being constructed will comprise a backbone or scaffold on which the multiple epitopes can be arranged and that the epitopes may be manufactured so as to be adjacent one another without an intervening scaffold portion or that the scaffold portion may be cleaved out of the protein sequence so that the end product may exist as a string of multiple relationally linked epitopes.

Preferably, the non-natural protein sequence of the invention comprises a plurality of epitopes, the number of epitopes may be more than 5 or 10 or 20 or 30 or 40 or 50 or more, there being no upper limit for their number rather the number being dictated by practical capabilities of their manufacture and a user's requirements.

Preferably, the epitopes may also function as a calibration entity in so far as an epitope may have a selected and known property such as and without limitation a known molecular weight.

It will be appreciated that the non-natural protein sequences of the present invention provide a product capable of serving simultaneously as a calibrant for two or more different biologically related components as well as providing a means of identification of the same.

Preferably, the epitopes are linked in series in a continuous length of sequence. The epitopes could be linked directly to one another or they could be attached to a backbone sequence comprising non-reactive or inert molecules.

In one embodiment of the invention, where the sequence allows, the epitopes could overlap. Again the epitopes could directly overlap one another or they may be interspersed with regions of an inert backbone sequence.

In an alternative embodiment of the invention, the epitopes could be discontinuous in the primary sequence of the product, that is to say some may be present at the N-terminus, others at the C-terminus and some may be present within the sequence of the scaffold portion.

It will be appreciated that in the instances that the epitopes are linked in series they are essentially non-branched non-natural protein sequences and as such there is no requirement for chemically reactive groups in the scaffold portion.

However in other embodiments, where the epitopes are not linked in series the one or more epitopes could preferably be covalently attached to the calibration portion at a site other than utilising the α-carbon backbone sequence. Such “branched” non-natural protein sequences could preferably be fabricated using covalent bonding through unique (or controlled numbers of) reactive residues such as cysteine, lysine, aspartate, glutamate, tyrosine.

Antibodies recognise relatively few amino acids in the particular protein target, which in one embodiment of the invention comprises a linear stretch of amino acids in the primary sequence (continuous epitope), and in another embodiment comprises amino acids in discontinuous in primary sequence, but contiguous in 3-dimensional space (discontinuous epitope).

In the present invention we use antibodies specific for individual epitopes of a number of relationally linked proteins or polypeptides in the non-natural protein sequence to understand the workings of, for example and without limitation a biochemical pathway. A quantitative understanding of such pathways is most desirable, and this also requires calibration of the output of studies using the protein/polypeptide specific antibodies. Since the non-natural protein sequence of the present invention also includes a calibration portion which comprises known amounts of epitope specific for each antibody in the series, it conveniently provides a means of investigating the quantitative relationships between components within a biochemical pathway or other series of proteins such as isoforms, allelic variations or proteins located within a common intra-cellular compartment.

The linking of a number of epitopes in series is an attractive advantage of the present invention as it reduces the cost of production of each product substantially (virtually in proportion with the number of epitopes in series) and thus makes the product profitable at reasonable price to the consumer.

Reference herein to a “scaffold portion” refers to a protein or concatamer within the non-natural sequence of the agent of the present invention which is non-reactive or innocuous and contributes to the calibration capability only in terms of its dominant physical properties for example by its molecular weight, and/or its pI, and/or its good production (expression) characteristics.

Preferably the scaffold portion of the non-natural protein sequence has a controlled property, preferably this property is relative molecular mass (Mr) or weight (Mwt) or the pH value for the isoelectric point of a given substance in solution (pI).

Preferably, the scaffold portion is a protein.

Preferably the scaffold portion comprises at least one natural or unnatural amino acid with at least one or more chemically reactive groups, preferably within the side chain of a residue.

Preferably the scaffold portion comprises one or more chemically reactive groups, for example, the carbonyl on glutamic acid or aspartic acid or the hydroxyl on tyrosine and more preferably still comprises at least one cysteine and/or lysine amino acid groups. Thus it will be appreciated that the scaffold portion may be a polymer containing a thiol or primary amine functional group or any other protein in which there are suitable reactive side chain groups such as aspartic acid, glutamic acid, cysteine and/or lysine groups available for covalent conjugation with the epitopes. It is desirable that the covalent conjugation of the epitopes to the chemically reactive groups of the scaffold portion be controlled.

Preferably, the number of reactive cysteine and/or lysine groups may be controlled by selecting the scaffold portion protein from a natural source which contains the desired number of reactive cysteine and/or lysine groups.

Preferably, the scaffold portion protein is selected from the group comprising: 127, from titin which contains two cysteine residues; I39 domain which is a subunit (subunit 5) of splicing factor 3b and which contains one cysteine residue, organ of Corti protein (Mus musculus) Swiss-Prot/TrEMBL Primary Accession Number Q8R448 which contains one cysteine and one lysine residue; heat shock protein, mitochondrial (Mus musculus) Swiss-Prot/TrEMBL Primary Accession Number Q64433 which contains eleven lysine residues; splicing factor 3B subunit 5 (Mus musculus) Swiss-Prot/TrEMBL Primary Accession Number Q923D4 which contains one cysteine and five lysine residues; ubiquinol-cytochrome C reductase complex ubiquinone-binding protein QP-C (Schizosaccharomyces pomme) Swiss-Prot/TrEMBL Primary Accession Number P50523 which contains one cysteine and six lysine residues; E1B protein (Human adenovirus type 11) Swiss-Prot/TrEMBL Primary Accession Number Q8B8U6 which contains one cysteine residue; chaperonin (Arabidopsis thaliana) Swiss-Prot/TrEMBL Primary Accession Number P34893 which contains nine lysine residues; photosystem 11 reaction centre H protein (Arabidopsis thaliana) Swiss-Prot/TrEMBL Primary Accession Number P56780 which contains three lysine residues; a NADH-ubiquinone oxidoreductase subunit, mitochondrial [Precursor] (Homo sapiens) Swiss-Prot/TrEMBL Primary Accession Number P56181 which contains one cysteine and nine lysine residues; signal recognition particle protein (Mus musculus) Swiss-Prot/TrEMBL Primary Accession Number P49962 which contains two cysteine and eight lysine residues; DNA polymerase delta subunit 4(Mus musculus) Swiss-Prot/TrEMBL Primary Accession Number Q9CWP8 which contains two cysteine and six lysine residues.

In a particular embodiment, the scaffold portion may comprise one or more domains, such as 127, from titin. Titin contains a number of β-sandwich domains belonging to the Ig family. The I27 domains usually contain two cysteine residues and fold to form stable structures of 10 kDa. In Nature, the I27 domain contains two cysteine residues (the site for covalent attachment of peptide); however mutation of these cysteine residues, to serine for example, is compatible with domain folding. Thus a presentation system of I27 can be formed where the molecular weight step size is a convenient unit (10 kDa steps) and where one unit (or more if required) can be engineered to possess a single cysteine residue for peptide attachment while all other units of I27 will lack cysteine residues. In alternative embodiments, the units of I27 may lack other reactive residues. These residues may include, but not be limited to lysine, glutamate and aspartate. A copy of I27 could contain one or more of these reactive residues, offering a controlled number of sites for the covalent attachment of epitopes.

Preferably, the number of reactive cysteine and/or lysine groups may be controlled by modifying any of the aforementioned scaffold portion proteins by selectively mutation by adding in or out or rendering ineffective any one or more of the reactive cysteine and/or lysine residues.

Alternatively, one or more of the titin domains may be mutated to possess either one or no cysteine residues. In one embodiment, the non-natural protein sequence comprises one or more I27 domains and a plurality of epitopes, wherein one of the I27 domains comprises a single cysteine residue and the other I27 domains lack a cysteine residue. In an alternative embodiment, the scaffold portion may comprise an I39 domain which is a subunit (subunit 5) of splicing factor 3b. The I39 domain is a 10 kDa domain. Preferably, the scaffold portion is of a convenient molecular weight and is typically selected as 10 kDa for convenience.

Preferably, the scaffold portion of the non-natural protein sequence of the present invention is blind to the antibody/antibodies specific to epitopes i.e. non-reactive thus, the scaffold portion may be considered capable of discrimination so that it is absolutely or relatively “immunologically blind” or “reactively inert” or substantially so.

Many proteins are covalently modified in a transient manner in response to a number of stimuli. This form of covalent modification can alter the function of the protein in question. Covalent modification can take the form of phosphorylation (of serine, threonine, tyrosine, lysine, arginine, histidine, aspartic acid and glutamic acid residues), sulphation of tyrosine residues, nitrosylation of cysteine residues (http://download.cell.com/supplementarydata/cell/106/6/675/DC1/TableS1.pdf) glycosylation (of threonine, serine residues), ubiquinatinylation and related modifications (of lysine residues). A full understanding of the process of transient covalent modification includes description of the proportion of molecules covalently modified at any particular point in time, this is termed the stoichiometry or modification and it represents the number of moles of modified protein per mole of total target protein (i.e. mol/mol).

In one embodiment of the invention the non-natural protein sequence of the present invention comprises at least two independent epitopes relating to a single protein.

Preferably, the at least two independent epitopes relating to a single protein are both situated on the backbone sequence comprising non-reactive or inert molecules or one may be situated there and the other associated with the scaffold portion.

The agents of the present invention can be used to determine the stoichiometry of modification of a particular site on a protein target. For example, total protein can be determined using a scaffold portion protein containing the epitope for the protein (where antibody binding to this epitope is not affected by any of the covalent modifications the protein can experience) as described above. In addition, the scaffold portion or concatamer portion can contain a single reactive residue to allow the covalent attachment of a second peptide epitope containing the covalent modification site, to which a modification-specific antibody exists. The non-natural protein sequence will thus contain two independent epitopes relating to this one protein, one (in series with the backbone of the scaffold protein) which records the total protein content of a sample, and a second (forming a branch in the polymer) peculiar to the covalent modification site. The use of this agent comprising a non-natural protein sequence with an experimental (test) sample will permit the collection of calibrated data relating to the binding of both antibodies to the agent and will facilitate calculation of the stoichiometry of modified proteins in the sample.

Preferably, the non-natural protein sequence comprises a single (or controlled number of) attachment site(s) for the second epitope, which will result in the creation of a branched polymer. Cysteine, lysine, aspartic acid, glutamic acid, tyrosine are the principal reactive residues found in proteins. “In line epitope” sequences (i.e. those continuous with the α-carbon backbone of the scaffold portion) will dictate whether the protein contains controlled numbers of these residues to facilitate attachment of the second epitope (modification specific) without corruption of the first (in line series). For example, where an epitope is being displayed by covalent attachment to a thiol group within the scaffold portion, the presence of thiol groups within the in line epitope(s) would be highly undesirable. In some cases the “in line epitope” sequence might prove incompatible with this approach, and alternative in line epitope sequences will be required.

In an alternative embodiment of the invention, the “branched” sequence may contain information for one or more epitope.

Preferably, multiple epitopes may be a contiguous sequence or in an overlapping sequence or they may contain hapten epitopes (e.g. phosphoamino acid, dyes, other modified amino acids) or alternatively they could be linear or branched structures.

For example, protein isoforms, generated from alternative genes, by alternative splicing, or by post-translational processing (e.g. proteolysis) can be used to create functional versatility from a limited genetic resource. Isoforms can differ in terms of their functional properties, their mode of regulation (partners, modification sites), their location in a cell, or alter their stability and life-time (1).

Thus it will be appreciated that the non-natural protein sequence of the present invention has the ability to bind partners specific to the selected epitopes and simultaneously to provide calibration qualities.

In a further aspect of the present invention, there is provided a non-natural protein sequence for use in detecting the presence of one or more relationally linked proteins or polypeptides in a sample and calibration of the said sample, the non-natural protein sequence product comprising a plurality of epitopes to proteins or polypeptides that are functionally related or linked and a scaffold portion comprising a non-reactive sequence that contributes to the calibration capability only in terms of its dominant physical properties as herein before described.

In a further aspect of the present invention, there is provided a non-natural protein sequence for use in detecting the presence of one or more relationally linked proteins or polypeptides and the absolute concentration of said epitope in a sample, the non-natural protein sequence product comprising a plurality of epitopes to proteins or polypeptides that are functionally related or linked and a scaffold portion comprising a non-reactive sequence that contributes to the calibration capability only in terms of its dominant physical properties as herein before described.

By determining the absolute concentration of an epitope in the sample we are able to equate this amount to a particular protein or protein modification.

In a further aspect of the invention, the non-natural protein sequence of the present invention can be used to calibrate a sandwich ELISA style experiment, wherein one antibody bound to a physical surface captures an antigen and then a second antibody, specific for a second feature on the same antigen, binds to the captured antigen. A schematic representation is presented in FIGS. 3 and 4. The immobilisation of the second antibody on the physical surface may then be detected using any of a number of means (calorimetric end point, chemiluminescent end point, fluorescent end point, radioactive end point). This experiment can in principle be performed in solution, and the end point measured using the diffusional characteristics of the second antibody. This antibody, labelled in a detectable way (e.g. with a fluorophore) will move more slowly when complexed to the antigen and first antibody than when it is present in solution alone. The change in rate can be detected by for example fluorescence correlation spectroscopy or fluorescence polarisation.

The present non-natural protein sequence or calibration material can incorporate multiple epitopes for antibodies specific for the same protein, spaced within the calibration product in such a way that more than one antibody can bind to its epitope simultaneously. In the example shown in FIG. 4, two epitopes from the same protein antigen (epitopes 1 and 2) are incorporated in the calibration product to allow capture of the product on a surface by interaction with antibody 1 (immobilised on the surface) and simultaneous interaction with antibody 2 (via epitope 2) leading to detection of bound antibody 2. Additional epitopes relating to the same protein target molecule may be incorporated in the calibration material (e.g. phospho-epitopes), and simultaneous binding of more than two antibodies to their epitopes could be achieved.

In a yet further aspect of the invention, there is provided a kit for identifying the presence of and quantifying the amount of at least two relationally linked proteins or polypeptides in a sample, the kit comprising a non-natural protein sequence product comprising a scaffold portion comprising a non-reactive sequence that contributes to the calibration capability only in terms of its dominant physical properties as herein before described and plurality of epitopes to proteins or polypeptides that are functionally related or linked. Optionally, the kit may comprise instructions for use thereof.

In a yet further aspect of the invention, there is provided a method of simultaneously detecting the presence of at least two relationally linked proteins or polypeptides and quantifying the amount of said functionally related or linked proteins or polypeptides in a sample, the method comprising:
a) providing a non-natural protein sequence, the sequence comprising a scaffold portion and at least two protein or polypeptide epitopes, the proteins or polypeptides being relationally linked and mixing this non-natural protein sequence with a test sample that may or may not contain binding partners to the epitopes;
b) adding at least two antibodies specific for the at least two epitopes;
c) carrying out a separation detection technique on said non-natural protein sequence; and optionally
d) generating at least one comparison point comprising intensity of a signal produced by the epitope portion of the non-natural protein sequence. Preferably, the method includes any one or more of the features hereinbefore described. The types of separation techniques that may be used in the method of the present invention are those as described in PCT/GB2005/00015.

The present invention is therefore of great utility to a researcher who may wish to study multiple related components in their system and at the same time provide a positive control, an internal standard or generate a calibration curve.

The present invention is of also great utility in industry pathway systems which can be altered to accomplish the manufacture of new products, those which occur in nature and others not normally produced in nature.

### BRIEF DESCRIPTION OF THE DRAWINGS

FIG. 1 shows a schematic representation of multiple epitopes in series.

FIG. 2 shows a schematic representation of a series of epitopes comprising isoforms of a single protein.

FIG. 3 shows a schematic representation of a sandwich ELISA assay.

FIG. 4 shows a schematic representation of the non-natural protein sequence of the present invention in use as a calibrant in a sandwich ELISA assay.

FIG. 5 shows cardiac ryanodine receptor and the sites of interaction of some of its associated proteins (Bers, D. M. (2004). Journal of Molecular and Cellular Cardiology, 37: 417-429).

FIG. 6 shows a cartoon block diagram of the protein expressed by E. coli., which contains the antibody epitopes for selected proteins involved in the Ryanodine receptor 2 (RyR2) macromolecular complex (Table 14: Notations 2-9).

FIG. 7 shows a cartoon diagram of the pGS-21a expression vector and the genetic sequence of the cloning region (GenScript).

FIG. 8 shows a Western blot of 12% SDS-PAGE gel of RyR2Macromolecular Complex Calibrant, loaded in doubling dilutions and with a uninduced control. Western blot probed with a rabbit anti-Calstabin2 (FKBP12.6) polyclonal primary antibody at 1 in 1000 dilution. Secondary antibody=GAR-HRP.

FIG. 9 shows a Western blot of the RyR2Macromolecular Complex Calibrant used to produce calibration plot—probed with a rabbit anti-Calstabin2 (FKBP12.6) polyclonal primary antibody and a secondary antibody=GAR-HRP.

FIG. 10 shows a calibration plot using the densitometry measured from the western blot in FIG. 9.

FIG. 11 shows a Western blot of the RyR2Macromolecular Complex Calibrant used to produce calibration plot—probed with a rabbit anti-PKA polyclonal primary antibody. Secondary antibody=GAR-HRP.

FIG. 12 shows a calibration plot using the densitometry measured from the western blot in FIG. 11.

FIG. 13 shows a cartoon block diagram of the protein expressed by E. coli., which contains the antibody epitopes for proteins involved in a MAPKinase pathway (Table 15: Notations 2-10).

FIG. 14 shows a cartoon diagram of the pGS-21a expression vector and the genetic sequence of the cloning region (GenScript).

FIG. 15 shows a Western blot of 12% SDS-PAGE gel of MAPKinase Pathway Calibrant, loaded in doubling dilutions and with a uninduced control. Western blot probed with a rabbit anti-EGFR polyclonal primary antibody at 1 in 500 dilution. Secondary antibody=GAR-HRP.

FIG. 16 shows a Western blot of the MAPKinase Pathway Calibrant used to produce calibration plot—probed with a rabbit anti-EGFR polyclonal primary antibody. Secondary antibody=GAR-HRP.

FIG. 17 shows a calibration plot using the densitometry measured from the western blot in FIG. 16.

FIG. 18 shows a Western blot of the MAPKinase Pathway Calibrant used to produce calibration plot—probed with a rabbit anti-Ras polyclonal primary antibody. Secondary antibody=GAR-HRP.

FIG. 19 shows a calibration plot using the densitometry measured from the western blot in FIG. 18.

FIG. 20 shows a Western blot of the MAPKinase Pathway Calibrant used to produce calibration plot—probed with a rabbit anti-ERK1+ERK2 polyclonal primary antibody. Secondary antibody=GAR-HRP.

FIG. 21 shows a calibration plot using the densitometry measured from the western blot in FIG. 20.

### MATERIAL AND METHODS

Antibodies specific for epitope sequences can be sourced from commercial or collaborative sources or produced using published procedures (Drago G. A., & Colyer, J. (1994) J. Biol. Chem. 269, 25073-25077; Hudson L., & Hay, F. C. (1980) Practical Immunology, 3^{rd} Ed., Blackwell Scientific Publications, Oxford). Conversely, the epitope of an antibody can be defined using published methods (Morris, G. L., Cheng, H-C., Colyer, J., & Wang, J. H. (1991) J. Biol. Chem. 266, 11270-11275).

### Design & Production of a Calibration Product for Relationally Linked Components:

The epitope of each antibody for a component in a relationally linked series must be established, either by virtue of the immunisation material (i.e. a peptide) or by empirical characterisation of the antibody. The peptide sequence of each chosen epitope (e.g. five sequences from different SERCA enzyme isoform in example 1) will be selected on the basis of their recognition by an appropriate antibody, and their chemical dissimilarity with other sequences in the calibration product. A gene encoding these peptide epitope sequences in series will be designed using codon usage information for Escherichia coli, or other relevant protein expression host. The gene (epitope gene) would be synthesised using published methods (Maniatis, Fritsch and Sambrook. 1st Ed 1982, 2nd Ed. 1989, Molecular Cloning: A laboratory manual. Cold Spring Harbor Press.).

The epitope gene was cloned into a further gene encoding the scaffold portion and an affinity purification tag, at an unique restriction site (Brockwell, D. J., Beddard, G. S., Clarkson, J., Zinober, R., Blake, A. W., Trinick, J., Olmsted, P. D., Smith, D. A., & Raadford, S. E. (2002) Biophys. J. 83, 458-472). Inserts with the correct orientation were identified by polymerase chain reaction using appropriate primer sets, and the gene product was expressed in E. coli as described in Brockwell et al. (2002).

The calibration product was purified by affinity chromatography (Brockwell et al., 2002) followed by preparative SDS-PAGE, and the amount of product was determined using a standard protein assay (Smith P K, Krohn R1, Hermanson G T, Mallia A K, Gartner F H, Provenzano M D, Fujimoto E K, Goeke N M, Olson B J, Klenk D C. Measurement of Protein Using Bicinchoninic Acid. Anal. Biochem. 1985; 150: 76-85)

### Calibration of Western Blot Experiments:

Known amounts of purified calibration product, in a serial dilution series from 10 pmol to 0.01 pmol were resolved on a 10% SDS-PAGE gel, transferred to PVDF membrane and stained with an antibody specific for the SERCA1a epitope. A series of biological samples containing SERCA proteins were analysed in parallel. Identical experiments were performed in series, or parallel and stained with antibodies specific for each of the other SERCA isoforms in turn, Immunosignals were quantified by densitometry (Rodriguez, P., Bhogal, M. S., & Colyer, J. (2003) J. Biol. Chem. 278, 38593-38600) and a calibration curve relating the immunosignal to calibrant loading (pmol) was used to determine the amount of epitope present in each biological sample.

### DETAILED DESCRIPTION

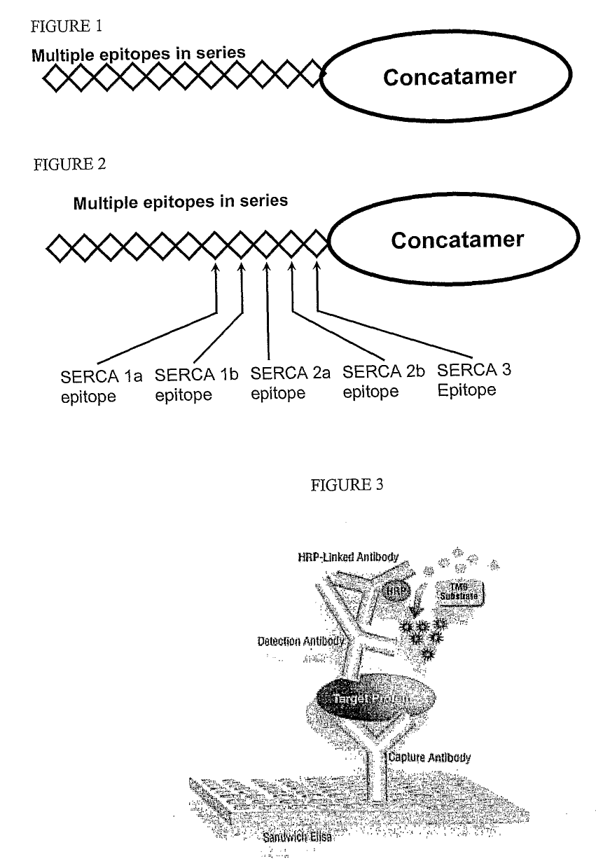

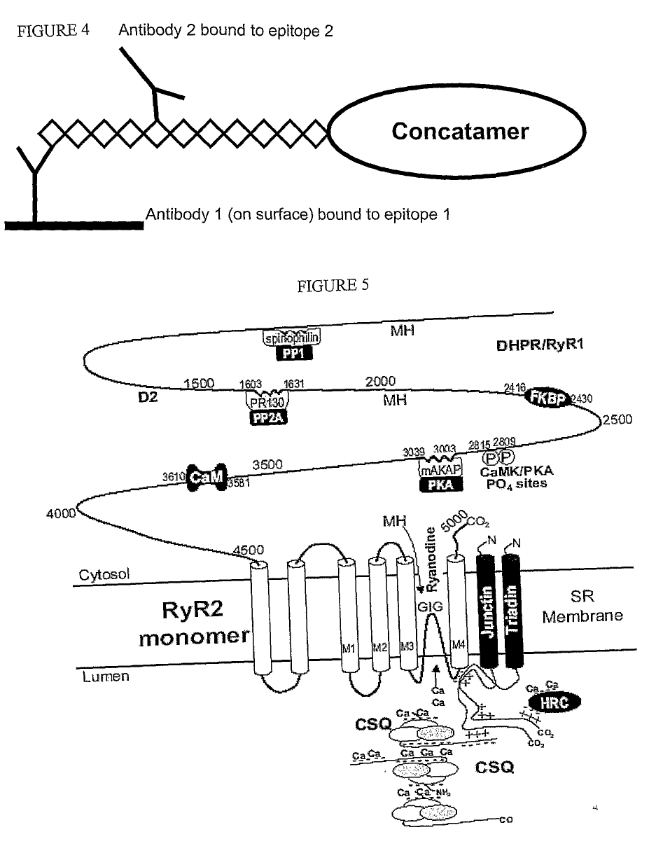

Taking, by way of example only a process A→B→C→D→E, catalysed by enzymes f, g, h, and i. Antibodies specific to each enzyme exist (called f′, g′, h′, and i′) and the epitope for each of these antibodies has been defined (f″, g″, h″, and i″). A calibration standard material could be constructed in which the chemical constituent of each antibody epitope (f″, g″, h″, and i″) is linked in series to form an unnatural protein sequence. This sequence is then linked in series to an additional protein sequence or scaffold portion, which is not recognised by any of the antibodies in this particular experimental series. This additional protein mass or scaffold portion functions in controlling the molecular weight of the non-natural protein sequence of the present invention. The non-natural protein sequence product will contain known amounts of each epitope, and can thus be used in experiments in known amounts to calibrate the signals generated by the experiment. The non-natural protein sequence of the present invention is shown schematically in FIG. 1, in which a series of 11 epitopes are linked in series to a scaffold portion also termed as an irrelevant protein or concatamer as in the Figure.

### EXAMPLE 1

### Isoforms

Many proteins can be expressed in a variety of isoforms, either from the expression of closely related genes or from the production of alternatively spliced forms of an individual gene, or by combination of both of these mechanisms.

The multifunctional SERCA Sarcoplasmic/endoplasmic reticulum (Ca²⁺—Mg²⁺)-Adenosine triphosphoatase exists in a number of isoforms generated from different genes (1,2,3), with alternative splicing products of genes SERCA1 and SERCA2 resulting in further diversity. See Table 1 below for details.

TABLE 1

| | | |
|---|---|---|
| Isoform | Molecular detail | Functional detail |

| | | |
|---|---|---|
| SERCA1a | FIARNYLE₉₉₄G₉₉₄ | Adult skeletal |
| | (SEQ ID NO:1) | isoform, fast- |
| | | twitch muscle (2) |
| SERCA1b | Excision of exon 42, | Foetal/neonatal |
| | C-terminal sequence | developmental |
| | FIARNYLE₉₉₄DPEDERRK₁₀₀₁ | expression |
| | (SEQ ID NO:2) | pattern, skeletal |
| | | muscle (3) |
| SERCA2a | LEP₉₉₄AILE₉₉₇ | Cardiac muscle |
| | (SEQ ID NO:3) | isoform (4) |
| SERCA2b | LEP₉₉₄GKECVQPATKSCSFSACT | Smooth muscle |
| | DGISWPFVLLIMPLVIWVYSTDTN | isoform (5) |
| | FSDMFWS₁₀₂₄ | |
| | (SEQ ID NO:4) which en- | |
| | codes a domain that | |
| | transverses the mem- | |
| | brane. | |
| SERCA3 | 75-77% identity with | Non-muscle isoform |
| | SERCA1 and SERCA2. | (primarily) (6) |
| | Sequence | |
| | ₉₉₀HHVDEKKDLk₉₉₉ | |
| | (SEQ ID NO:5) is iso- | |
| | form specific | |

With reference to FIG. 2, there is shown a schematic representation of a non-natural protein sequence of the present invention which can be used to investigate isoforms of SERCA Sarcoplasmic/endoplasmic reticulum (Ca²⁺—Mg²⁺)-Adenosine triphosphoatase

### EXAMPLE 2

### Isoforms

The multifunctional protein phosphatase, calcineurin (CaN) is an example of an enzyme expressed in a variety of isoforms. CaN is involved in a large variety of biological events including programmes of gene expression in response to extracellular signals (CaN/NFAT). The calcineurin isoforms are as follows: Calcineurin alpha (CaN alpha); Calcineurin beta (CaN beta); and Calcineurin gamma (CaN gamma) sequence not commercially available. A product of the invention comprises the three calcineurin isoforms and Table 2 below for details.

TABLE 2

| | | |
|---|---|---|
| Antibody | | Cross |
| for: | Epitope | Reactivity |

| | | | |
|---|---|---|---|
| 1 | CaN alpha | Synthetic peptide (CDPKLSTTDRVV) | All |
| | | SEQ ID NO:6 coupled to KLH | mammals |
| 2 | CaN beta | 25 residue synthetic peptide | Bovine, |
| | | corresponding to amino acids | Human, |
| | | 500-524 SEQ ID NO:7 | Mouse, |
| | | (VQQDGFNSLNTAHTTENHGTGNHSA) of | Rat. |
| | | rat Calcineurin/PP2B Aβ | |

| | | |
|---|---|---|
| 3 | CaN gamma | Antibodies to this protein with published epitope |
| | | characteristics are not currently available from |
| | | commercial sources. An antibody might be available |
| | | by collaboration with an academic group, wherein |
| | | the epitope of the antibody would be known, or |
| | | could be discovered. Alternatively a new antibody |
| | | could be created to an immunogenic sequence |
| | | peculiar to this protein, and this sequence could |
| | | be included in the calibration product |

### EXAMPLE 3

### Isoforms

The multifunctional protein kinase, calmodulin-dependent kinase II is an example of an enzyme expressed in a variety of isoforms. CaM kinase II is involved in a large variety of biological events including memory, regulation of vesicle movement, and maladaptive responses in heart failure. The isoforms of CAMKII are listed below:

1. CaMKII alpha,
(SEQ ID NO:8 KWQIVHFHRSGAPSVLPH)
2. CamKII beta
(SEQ ID NO:9 RRGSGTPEAEAPRQW)
3. CamKII delta
(SEQ ID NO:10 QNVHFHRSGSPTVPIK)
4. CamKII gamma
(SEQ ID NO:11 RSPEGRSSRDRTAPS)
5. CamKII antibody
(SEQ ID NO 12 CTRFTDEYQLFEEL)
6. CamKII alpha phospho (T286)
(SEQ ID NO 13: MHRQET(PO₄)VDCLKKFN)

A calibration product could be constructed from a series of epitope sequences, where each sequence represents the epitope for an antibody specific for an isoform of Cam kinase II. Some epitopes are shared between all or several isoforms, these epitopes could be incorporated in the calibration standard to calibrate multiple isoforms with a single antibody (e.g. module 5). A number of phosphorylation sites exist in the protein. Epitopes for phosphorylation site specific antibodies could be incorporated in the product (e.g. module 6 above) to calibrate the status of phosphorylation too. Details of the antibodies and epitopes are set out below in Table 3.

### EXAMPLE 4

### Polymorphisms

Polymorphisms occur within biological species in probably every gene. In some cases the polymorphisms occur with altered probability in disease situations, and in those case are of particular interest and use.

Genetic variation exists within the population of a species, which at the individual gene level is manifest as polymorphisms of a gene. Polymorphisms represent typically single base changes in the sequence of a gene which can occur in the coding or non-coding regions. These deviations in sequence can be without consequence to the gene, or can alter the level of expression of the gene, or can alter the polymer encoded by the gene. In many instances the probability of disease is linked to particular polymorphisms, which serves as a useful screening tool, and as a basis for hypothesis driven research into the cause and management of disease.

TABLE 3

| | | |
|---|---|---|
| Antibody | Epitope | Specificity |

| | | | |
|---|---|---|---|
| 1 | CAMK2 alpha Antibody (C- | KWQIVHFHRSGA | Human |
| | term) (Purified Rabbit | PSVLPH (from | |
| | Polyclonal Antibody) | Human) | |
| | | SEQ ID NO:8 | |
| 2 | CAMK2 beta Antibody (C- | RRGSGTPEAEAPRQW | Human, |
| | term) (Purified Rabbit | (from human) | Primate |
| | Polyclonal Antibody) | SEQ ID NO:9 | |
| 3 | CAMK2 delta Antibody (C- | QNVHFHRSGSPTVPIK | Human, |
| | term) (Purified Rabbit | (from human) | Primate |
| | Polyclonal Antibody) | SEQ ID NO:10 | |
| 4 | CAMK2 gamma Antibody (C- | RSPEGRSSRDRTAPS | Human |
| | term) (Purified Rabbit | (from human) | |
| | Polyclonal Antibody) | SEQ ID NO:11 | |
| 5 | CAMK2 Antibody (N-term) | CTRFTDEYQLFEEL | This antibody recognizes |
| | (Purified Rabbit Poly- | (from Rat delta | alpha, delta, and gamma |
| | clonal Antibody) | isoform) | (NOT beta) isoforms of |
| | | SEQ ID NO:12 | human, mouse, rat, |
| | | | bovine, hamster, guinea |
| | | | pig, chicken, and rabbit |
| | | | CaMKII |
| 6 | CaMKII alpha (phospho | MHRQET(PO₄)VDCLKK | Rat |
| | T286) antibody | FN (from Rat) | |
| | (monoclonal) | SEQ ID NO:13 | |

Certain polymorphisms in the RYR2 gene, which encodes an ion channel expressed in the heart, are associated with a disorder known as catecholaminergic polymorphic ventricular tachycardia (CPVT) which can provoke electrical irregularity and sudden death when an individual exercises. To date, over 20 separate mis-sense polymorphisms (those which alter the primary sequence of the protein) have been discovered in the human RYR2 gene, which are linked to CPVT. These include:

Mutation (ref 6)

S2246L
E2311D
R2474S
L3778F
G3946S
N4104K
R4497C
V4771I
A4860G
I4867M
N4895D
E4950K

Which are residues conserved between man (and mouse), Drosophila and Caenorhabditis elegans. They exist in regions which are also highly conserved, both across these species, and between isoforms of RYR (1,2, and 3).

### EXAMPLE 5

### Polymorphisms

A large number of mutations in RyR2 are found in patients with arrhythmogenic right ventricular dysplasia type 2 (ARVD2) and catecholaminergic polymorphic ventricular tachycardia (CPVT). These are believed to play a causal role in disease. A sub-set of known disease associated mutations of RYR2 include: (1) R176 Q, (2) V2306 I, (3) G3946 S and (4) V4653 F. To our knowledge antibodies specific for these mutations do not exist, however it is likely that they can be generated using short synthetic peptide immunogen incorporating the mutation site, using techniques known in the art. Calibration of such antibodies could be achieved using a product comprising SEQ ID NOs 19-22 (see Table 4 below).

TABLE 4

| | | | |
|---|---|---|---|
| Antibody | | | Epitope after |
| for: | Domain | Pre-mutation | mutation |

| | | | | |
|---|---|---|---|---|
| 1 | R 176 Q | N- | EGEKVRVGDDL | EGEKVQVGDDL |
| | | terminal | (SEQ ID NO: 14) | (SEQ ID NO: 18) |
| 2 | V2306 I | Central | FLRFAVECNGE | FLRFAIFCNGE |
| | | | (SEQ ID NO: 15) | (SEQ ID NO: 19) |
| 3 | G3946 S | C- | WDAVVGFLHVF | WDAVVSFLHVF |
| | | terminal | (SEQ ID NO: 16) | (SEQ ID NO: 20) |
| 4 | V4653 F | C- | FVKRKVMDKYG | FVKRKFMDKYG |
| | | terminal | (SEQ ID NO: 17) | (SEQ ID NO: 21) |

### EXAMPLE 6

### Polymorphisms

Protein p53 (also called TP53) is associated with a high proportion of cancers in man. For example in human liver cancer, 26% of cases (559 of 2153 tumours) show mutation in TP53 according to 64 studies (Jackson et al; 2006 Toxicology Science 90, 400-418). Similarly TP53 mutations occur in 42% of spontaneous lung tumours in man. Missense polymorphisms result in mutant proteins, some of which are associated with cancer, such as: (1) R 249 S—most frequent TP53 mutation in hepatocellular carcinoria (HCC), (2) R172P, (3) R172H and (4) R270H. To our knowledge antibodies specific for these mutations do not exist, however it is likely that they can be generated using short synthetic peptide immunogen incorporating the mutation site, using techniques known in the art. Calibration of such antibodies could be achieved using the following product comprising SEQ ID NOs 26-29 of the epitopes after mutation, see Table 5 below:

TABLE 5

| | | | |
|---|---|---|---|
| Antibody | Type of | | Epitope after |
| for: | TP53 | Pre-mutation | mutation |

| | | | | |
|---|---|---|---|---|
| 1 | R 249 S | Human | GGMNRRP ILTI | GGMNRSP |
| | | | (SEQ ID NO:22) | ILTI |
| | | | | (SEQ ID NO:26) |
| 2 | R 172 P | Mouse | TEVV RRCPHHE | TEVV |
| | | | (SEQ ID NO:23) | RPCPHHE |
| | | | | (SEQ ID NO:27) |
| 3 | R 172 H | Mouse | TEVV RRCPHHE | TEVV |
| | | | (SEQ ID NO:24) | RHCPHHE |
| | | | | (SEQ ID NO:28) |
| 4 | R 270 H | Mouse | DSFEVR VCACP | DSFEVH |
| | | | (SEQ ID NO:25) | VCACP |
| | | | | (SEQ ID NO:29) |

### EXAMPLE 7

### Components with a Functional Relationship; Pathways

Proteins are involved in natural processes such as metabolism, blood clotting, and hormone action. For example, glycolysis: achieves the following chemical reaction

C₆H₁₂O₆+2ADP+2Pᵢ+2NAD⁺→2CH₃COCOO+2ATP+2H₂O+2NADH+2H⁺

It utilises 10 enzymes, and an 11^{th} under anaerobic conditions, specifically the enzymes are as follows:

Hexokinase, Phosphoglucose isomerase, 6-phosphofructose-1-kinase, fructose bisphosphate aldolase, triose phosphate isomerase, glyceraldehydes 3-phosphate dehydrogenase, phosphoglycerate kinase, phosphoglycerate mutase, enolase, pyruvate kinase, (lactate dehydrogenase).

A calibration standard comprising epitopes for antibodies to each of these proteins would be useful in the study of glycolytic processes in biology, biotechnology and medicine.

### EXAMPLE 8

### Components with a Functional Relationship; Pathways

The eukaryotic cell cycle is an essential pathway necessary for all proliferative responses. This cycle involves a number of protein kinases and their partner regulatory proteins (cyclins), the concentration of the cyclins change throughout the cell cycle to allow passage of the cell through specific controlling check-points. Cell cycle engine parts include: (1) Cyclin A; (2) cdK1; (3) Cyclin D; (4) cdK4; (5) cdK6; (6) Cyclin E; (7) cdK2; and (8) Cyclin B. A product comprising the non-natural protein sequence comprising two or more epitopes selected from 1-8 as defined in Table 6 below would be useful in the study of cell cycles in biology, biotechnology and medicine.

| | | |
|---|---|---|
| Antibody for: | Epitope | Cross Reactivity |

| | | | |
|---|---|---|---|
| 1 | Cyclin A | Synthetic peptide: C- | Human, Mouse, Rat |
| | | KYHGVSLLNPPETLNL, (SEQ ID | |
| | | NO:30) corresponding to amino acids | |
| | | 417-432 of Human Cyclin A | |
| 2 | cdK1 | Recombinant fragment (Xenopus | Human, Mouse, Rat, |
| | | laevis). C-Terminal 2/3rds of Xenopus | Xenopus laevis, |
| | | cdc2 expressed in E. coli (220-227) | Chicken, Guinea pig |
| | | | and Woodchuck |
| 3 | Cyclin D | peptide corresponding the 11 C- | Human, Mouse, Rat |
| | | terminal amino acids, residues 285- | |
| | | 295, of human Cyclin D1 | |
| | | (CTPTDVRDVDI), (SEQ ID NO:31) | |
| 4 | cdK4 | MATSRYEPVA EIGVGAYGTV (From | Human, Rat, Mouse, |
| | | Human), (SEQ ID NO:32) | Pig |
| 5 | cdK6 | Synthetic peptide: C-SQNTSELNTA, | Human, Mouse, Rat |
| | | (SEQ ID NO:33) corresponding to | |
| | | amino acids 317-326 of Human Cdk6 | |
| 6 | cyclin E | Synthetic peptide (amino acid | Human, Mouse |
| | | sequence considered to be | |
| | | commercially sensitive) (Human) (C | |
| | | terminal | |
| 7 | cdK2 (cdc2) | The epitope is thought to be residues | Human, Rat, Mouse, |
| | | 220-227 of mouse cdc2, LGTPNNEV, | Chicken, Xenopus |
| | | (SEQ ID NO:34) | laevis and |
| | | | Amphibians |
| 8 | Cyclin B | generated from 14-168 of cyclin B | Human, Rat |
| | | (human) | |

### EXAMPLE 9

### Components with a Functional Relationship; Pathways

A series of components acting in consort can form a pathway. Large numbers of pathways exist in biochemistry. One example is a pathway of interactions which control the expression of cell cycle regulators, cyclin A and cyclin E. G1 cyclins that overcome inhibitors of cell cycle progression are: (1) P16; (2) Cyclin D; (3) Retinoblastoma Protein; (4) E2F; (5) Cyclin E; (6) Cyclin A; and (7) P27. A product comprising the non-natural protein sequence comprising two or more epitopes selected from 1-7 as defined in Table 7 below would be useful in the study of cell cycles in biology, biotechnology and medicine.

TABLE 7

| | | |
|---|---|---|
| Antibody | | |
| for: | Epitope | Cross Reactivity |

| | | | |
|---|---|---|---|
| 1 | P16 | human p16 protein (missing the first 7 amino | Human. Cross |
| | | acids) SME PSADWLATAA ARGRVEEVRA | reactivity may occur |
| | | LLEAGALPNA PNSYGRRPIQ VMMMGSARVA | with p15 when |
| | | ELLLLHGAEP NCADPATLTR PVHDAAREGF | immunoprecipitating |
| | | LDTLVVLHRA GARLDVRDAW GRLPVDLAEE | in vitro translated |
| | | LGHRDVARYL RAAAGGTRGS NHARIDAAEG | product but not in |
| | | PSDIPD (SEQ ID NO:35) | immunoblots. |
| | | | Potentially cross |
| | | | reacts with p15.5. |
| 2 | Cyclin D | peptide corresponding the 11 C-terminal amino | Human, |
| | | acids. residues 285-295, of human Cyclin D1 | Mouse, Rat |
| | | (CTPTDVRDVDI) (SEQ ID NO:36) | |
| 3 | Retino- | Antibodies to this protein with published epitope | Human |
| | blastoma | characteristics are not currently available from | |
| | Protein | commercial sources. An antibody might be | |
| | | available by collaboration with an academic group, | |
| | | wherein the epitope of the antibody would be | |
| | | known, or could be discovered. Alternatively a new | |
| | | antibody could be created to an immunogenic | |
| | | sequence peculiar to this protein, and this | |
| | | sequence could be included in the calibration | |
| | | product | |
| 4 | E2F | SPPPSSPPS SLTTDPSQSL LSLEQEPLLS | Human |
| | | RMGSLRAPVD EDRLSPLVAA DSLLEHVRED (from | |
| | | human E2F-1)(SEQ ID NO:37) | |
| 5 | Cyclin E | Antibodies to this protein with published epitope | Human, |
| | | characteristics are not currently available from | Mouse |
| | | commercial sources. A new antibody could be | |
| | | created to an immunogenic sequence peculiar to | |
| | | this protein, and this sequence could be included | |
| | | in the calibration product | |
| 6 | Cyclin A | Synthetic peptide: C-KYHGVSLLNPPETLNL, (SEQ ID | Human, |
| | | NO:38)corresponding to amino acids 417-432 of | Mouse, Rat |
| | | Human Cyclin A | |
| 7 | p27 | MSNVRVSNGS PSLERMDARQ AEHPKPSACR | Human, |
| | | NLFGPVDHEE LTRDLEKHCR DMEEASQRKW | Mouse, Rat |
| | | NFDFQNHKPL EGKYEWQEVE KGSLPEFYYR | |
| | | PPRPPKGACK VPAQESQDVS GSRPAAPLIG | |
| | | APANSEDTHL VDPKTDPSDS QTGLAEQCAG | |
| | | IRKRPATDDS STQNKRANRT EENVSDGSPN | |
| | | AGSVEQTPKK PGLRRRQT (SEQ ID NO:39) | |

### EXAMPLE 10

### Components with a Functional Relationship; Pathways

An example of a pathway of interactions which communicate extracellular stimuli to changes in gene expression, involving NFAT and calcineurin is: (1) CHP; (2) FK506; (3) MCIP/calcipressin; (4) AKAP79; (5) CsA/CyA; (6) Cabin 1/CAIN; (7) NFAT(P); (8) PKA; (9) CKI; (10) GSK-3beta; (11) JNK; (12) P38; (13) MEF2; and (14)NFAT. Table 8 below shows the details for the manufacture a non-natural protein sequence of the present invention using two or more epitopes selected from the group comprising epitopes 1-14.

TABLE 8

| | | |
|---|---|---|
| Antibody | | |
| for: | Epitope | Cross Reactivity |

| | | | |
|---|---|---|---|
| 1 | CHP | MESHSVTQAGVQWRDLGSLQPLPPGFKQFSHLSL | Human |
| | | PSSWDYRRVPPYLGNFCIFSGEGVSPCWPGWS (SEQ | |
| | | ID NO:40) | |
| 2 | FK506 | Antibodies to this protein with published epitope | Recognizes |
| | | characteristics are not currently available from | conjugated and |
| | | commercial sources. A new antibody could be | non-conjugated |
| | | created to an immunogenic sequence peculiar to | FK-506 with |
| | | this protein, and this sequence could be included | apparent |
| | | in the calibration product | dissociation |
| | | | constant |
| | | | Kd = 10-9 M |
| 3 | MCIP/ | Synthetic peptide: EEVDLQDLPSAT(SEQ ID | Human and |
| | calci- | NO:41), corresponding to N terminal amino acids | Mouse |
| | pressin | 2-13 of Human Calcipressin 1 | |
| 4 | AKAP79 | Sythetic peptide produced from human AKAP79 | Human |
| | | aa180-427 | |
| | | QDLSEGISQKDGDEVCESNVSNSITSGEKVISVELGLDN | |
| | | GHSAIQTGTLILEEIETIKEKQDVQPQQASPLETSETDH | |
| | | QQPVLSDVPPLPAIPDQQIVEEASNSTLESAPNGKDYES | |
| | | TEIVAEETKPKDTELSQESDFKENGITEEKSKSEESKRM | |
| | | EPIAIIITDTEISEFDVTKSKNVPKQFLISAENEQVGVF | |
| | | ANDNGFEDRTSEQYETLLIETASSLVKNAIQLSIEQLVN | |
| | | EMASDDNKINNLLQ (SEQ ID NO:42), | |
| 5 | CsA/CyA | Full length human recombinant cyclophilin A | Human, Mouse, |
| | | MVNPTVFFDITADDEPLGRVSFELFADKVPKTAENFRAL | Rat |
| | | STGEKGFGYKGSSFHRIIPGFMCQGGDFTRHNGTGGRSI | |
| | | YGEKFEDENFILKHTGPGILSMANAGPNTNGSQFFICTA | |
| | | KTEWLDGKHVVFGKVKEGMNIVEAMERFGSRNGKTSKKI | |
| | | TISDCGQL (SEQ ID NO:43) | |
| 6 | Cabin | MIRIAALNASSTIEDDHEGSFKSHKTQTKEAQEA | Human |
| | 1/CAIN | EAFALYHKALDLQKHDRFEESAKAYHELLEASLL | |
| | | REAVSSGDEKEGLKHPGLILKYSTYKNLAQLAAQ | |
| | | REDLETAM (SEQ ID NO:44) | |

| | | |
|---|---|---|
| 7 | NFAT(P) | Antibodies to this protein with published epitope characteristics |
| | | are not currently available from commercial sources. A new |
| | | antibody could be created to an immunogenic sequence peculiar to |
| | | this protein, and this sequence could be included in the |
| | | calibration product |

| | | | |
|---|---|---|---|
| 8 | PKA | KKGSEQESVKEFLAK (SEQ ID NO:45), (aa 8-22 | Human, Bovine, |
| | | human alpha isoform of the catalytic subunit) | Mouse, Porcine, |
| | | | Rat |
| 9 | CKI | synthetic peptide selected from aa 324~337 of | Human |
| | | human CK1a | |
| 10 | GSK- | Rat GSK3 beta synthetic peptide (aa. 335-349)1, | Bovine, Human, |
| | 3beta | conjugated to KLH; identical to human GSK3 beta | Mouse, Rat |
| | | sequence over these residues (CAHSFF | |
| | | DELRDPNVK) (SEQ ID NO:46) | |
| 11 | JNK | Fusion protein: HIDQWNKVIEQ LGTPSAEFMKK | Human, Mouse |
| | | LQPTVRNYVEN RPKYPGIKFEE LFPDWIFPSES | and Rat |
| | | ERDKIKTSQAR DLLSKMLVIDPD KRISVDEALRHP | |
| | | YITVWYDPAEAE APPPQIYDAQ LEEREHAIEEW | |
| | | KELIYKEVMDWE ERSKNGVVKDQ PSDAAVSSNATP | |
| | | SQSSSINDISSMST EQTLASDTDSSLD ASTGPLEGCR | |
| | | (SEQ ID NO:47), corresponding to amino acids 231-424 | |
| | | of Human JNK2 | |
| 12 | p38 | Synthetic peptide: TYDEVISFVPPPLDQEEMES, (SEQ | Human, Mouse, |
| | | ID NO:48) corresponding to amino acids 341/360 | Rat, Rabbit, |
| | | of Human p38 | Cow, Chicken, |
| | | | Dog, Guinea |
| | | | pig, Hamster, |
| | | | Monkey, Pig |
| | | | and Sheep |
| 13 | MEF2 | Synthetic peptide derived from a region between | Human |
| | | residues 227-276 of human Myelin expression | |
| | | factor 2 (QAGR LGSTIFVANL DFKVGWKKLK | |
| | | EVFSIAGTVK RADIKED) (SEQ ID NO:49) | |
| 14 | NFAT | Synthetic peptide: | Human, Rat |
| | | (51)AISSPSGLAYPDDVLDYGL(69).(SEQ ID NO:50) | and Mouse |
| | | The immunizing peptide corresponds to amino acid | |
| | | residues 51-69 of mouse NFAT1-A, B and C | |
| | | isoforms | |

### EXAMPLE 11

### Components with a Functional Relationship; Pathways

A further example pathway of interactions is control of the production of cytokines downstream of the toll-like receptor. Lipopolysaccharide is a ligand for the Toll-like receptor and the proteins involved in the signalling network include: (1) TLR; (2) TRIF; (3) TRAF6; (4) JNK; (5) IL10; (6) IL6; (7) RANTES; (8) GCSF; (9) TNF alpha; and (10) MIPI alpha. A product comprising the non-natural protein sequence comprising two or more epitopes selected from 1-10 as defined in Table 9 below would be useful in the study of cytokine production in biology, biotechnology and medicine.

| | | |
|---|---|---|
| Antibody | | Cross |
| for: | Epitope | Reactivity |

| | | | |
|---|---|---|---|
| 1 | TLR | Amino acids 215-410 of TLR2 (VIRDLL HSAIWLDVRK | Human |
| | | LAFSVPEKIQLLRIMSSSFA KKISLKQCLF TDATVPEIVS | |
| | | ILEGMPKLME VEMKDCTLLG | |
| | | TGKWYKQIHANQSQSLRILT IENLSIEEFY | |
| | | LFTDLQSVLD LLSLFRKVTV ENTKVFLVPC | |
| | | KLSQHLLSLEYLDLSANLLG DQSLEHSACQ | |
| | | GAWPSLQTLN LSQNSLSDLK MTGKSLFHLR) (SEQ ID | |
| | | NO: 51) | |
| 2 | TRIF | Synthetic peptide, corresponding to amino acids 143-158 | Human and |
| | | of Mouse TRIF (ERGLSGLFRN LTLERVVE) (SEQ ID | Mouse |
| | | NO: 52) | |
| 3 | TRAF6 | Synthetic peptide: DQSEAPVRQNHEEI, (SEQ ID NO: 53) | Human and |
| | | corresponding to amino acids 436-449 of Human TRAF6 | Mouse |
| 4 | JNK | Fusion protein: HIDQWNKVIEQ LGTPSAEFMKK | Human, Mouse |
| | | LQPTVRNYVEN RPKYPGIKFEE LFPDWIFPSES | and Rat |
| | | ERDKIKTSQAR DLLSKMLVIDPD KRISVDEALRHP | |
| | | YITVWYDPAEAE APPPQIYDAQ LEEREHAIEEW | |
| | | KELIYKEVMDWE ERSKNGVVKDQ PSDAAVSSNATP | |
| | | SQSSSINDISSMST EQTLASDTDSSLD ASTGPLEGCR, | |
| | | (SEQ ID NO: 54) corresponding to amino acids 231-424 | |
| | | of Human JNK2 | |
| 5 | IL10 | Fragment, corresponding to amino acids 19-178 of | Human |
| | | Human IL10 (ALLSLRLGSD AHGTELPSPP | |
| | | SVWFEAEFFH HILHWTPIPN QSESTCYEVA (SEQ ID | |
| | | NO: 55) | |
| 6 | IL6 | COS-7-expressed, recombinant mouse IL-6 | Mouse |
| 7 | RANTES | Synthetic peptide: | Human, Mouse |
| | | SPYSSDTTPCCFAYIARPLPRAHIKEYFYTSGKCSNPAV | and Rat |
| | | VFVTRKNRQVCANPEKKWVREYINSLEM, (SEQ ID NO: | |
| | | 56) corresponding to amino acids 24-90 of Human | |
| | | RANTES. | |
| 8 | GCSF | Recombinant full length protein, express in mouse NSO | Human |
| | | cells | |
| 9 | TNF | Highly pure (>98%) recombinant full length protein | Mouse |
| | alpha | (Mouse) | |
| 10 | MIP1 | Synthetic peptide: | Human |
| | | CVETFGTTSYYDDVGLLCEKADTRALMA (SEQ ID NO: | |
| | | 57)(Human) | |

### EXAMPLE 12

### Proteins Sharing a Common Location in the Cell

Some biological components share a common location in a cell for all or some of their time. A number of signals contained within the primary sequence of proteins control their location in the cell. Residence of this location is typically dynamic (rather than static) and thus evaluation of the entire protein complement of that location would be valuable in biological research.

Lipid raft domains of biological membranes are an interesting example of a discrete cellular location. Our present understanding places lipid rafts as subdomains of the plasma membrane, characterised by a gel phase lipid composition (lipid and cholesterol) which allows residence of some particular proteins and exclusion of others. Three distinct lipid raft types can be resolved, as summarised in table below (taken from http://www.bms.ed.ac.uk/research/others/smaciver/Cyto-Topics/lipid rafts and the cytoskeleton.htm):

| Raft Type | Constituent | Properties | Function |
|---|---|---|---|
| Caveolae | Cholesterol, glycol- | Invagina- | Presumed to |
| | sphingolipid, Arachidonic | tions of | be signalling |
| | acid, Plasmenylethanol- | membranes | centres and |
| | amine, Caveolin1 and 2, | rich in | perhaps |
| | hetero- trimeric G- | signalling | regions of |
| | proteins and monomeric | molecules | cholesterol |
| | G-proteins, EGF & PDGF | | import |
| | receptors, Fyn, GPI- | | |
| | linked enzymes, integrins. | | |
| | Flotillin | | |
| Glycosphingo- | Cholesterol, glycoshingo- | Detergent | Signalling |
| lipid enriched | lipid, low in PI and other | resistant | |
| | anionic phospholipids | membranes | |
| PIP₂ enriched | PIP₂, MARKS, CAP, | | Signalling, |
| | GAP-43 | | Structural. |

### EXAMPLE 13

### Proteins Sharing a Common Location in the Cell

The centrosome is a common physical location for some biological components. It is located adjacent to the eukaryotic nucleus and serves a variety of functions including the organisation of microtubules. The centrosome organises the assembly of the mitotic spindle which permits the correct segregation of chromosomes. Abnormalities in centrosome components can lead to centrosome dysfunction, which is often associated with proliferative diseases such as cancer. Centrosomes also play important roles in cell migration, the movement of cilia and the movement of vesicular membrane structures within cells. Centrosome contains a number of proteins, including: (1) Microtubule; (2) Pericentrin; (3) Centrin; (4) PCMI; (5) Ninein; (6) BBS4; (7) P150 Glued; (8) Dynein; (9) Centriolin; (10) Gamma Tubulin; (11) Polo Kinases; (12) Aurora Kinases; (13) Catanin; and (14) Katanin.

A product comprising the non-natural protein sequence comprising two or more epitopes selected from 1-14 as defined in Table 10 below would be useful in the study of cell cycles in biology, biotechnology and medicine. Furthermore a calibration product comprising amino acid sequences to antibodies specific to multiple components below is envisaged. In some instances suitable antibodies with known epitope sequences have been described, in other instances such antibodies need to be identified.

TABLE 10

| | Antibody | | |
|---|---|---|---|
| | for: | Epitope | Cross Reactivity |
| 1 | Mircotubule | K562 erythroleukemia cell line | Human |
| 2 | pericentrin | aa 142-592 of mouse pericentrin. It is expressed | Human |
| | | as a GST fusion protein. The corresponding amino | |
| | | acids are present in both pericentrin and kendrin | |
| 3 | Centrin | synthetic peptide corresponding to the C-terminus | Human |
| | | of human centrin 1 (amino acids 157-172) | |
| 4 | PCM1 | Antibodies to this protein with published epitope characteristics are | |
| | | not currently available from commercial sources. A new antibody | |
| | | could be created to an immunogenic sequence peculiar to this | |
| | | protein, and this sequence could be included in the calibration | |
| | | product | |
| 5 | Ninein | Fusion protein containing amino acids 32-506 of | Human. |
| | | murine Ninein | Predicted to |
| | | | react with |
| | | | Mouse due to |
| | | | sequence |
| | | | homology |
| 6 | BBS4 | Antibodies to this protein with published epitope characteristics are | |
| | | not currently available from commercial sources. An antibody might | |
| | | be available by collaboration with an academic group, wherein the | |
| | | epitope of the antibody would be known, or could be discovered. | |
| | | Alternatively a new antibody could be created to an immunogenic | |
| | | sequence peculiar to this protein, and this sequence could be | |
| | | included in the calibration product | |
| 7 | p150 | Synthetic peptide produced from aa3-202 of rat | Chicken, Dog, |
| | Glued | p150Glued | Human, Mouse, |
| | | | Rat |
| 8 | Dynein | Cytoplasmic full length native protein (purified from | Human and |
| | | chick brain) (Chicken) | Chicken |
| 9 | Centriolin | Antibodies to this protein with published epitope characteristics are | |
| | | not currently available from commercial sources. An antibody might | |
| | | be available by collaboration with an academic group, wherein the | |
| | | epitope of the antibody would be known, or could be discovered. | |
| | | Alternatively a new antibody could be created to an immunogenic | |
| | | sequence peculiar to this protein, and this sequence could be | |
| | | included in the calibration product | |
| 10 | gamma | Synthetic peptide: EEFATEGTDRKDVFFY-C | Reacts with |
| | Tubulin | (SEQ ID NO: 58) conjugated to KLH, | Human, |
| | | corresponding to C terminal amino acids 38-53 of | Chicken and |
| | | Human gamma Tubulin | Hamster |
| 11 | Polo | aa330-370 OF His-Plk1 | Human and |
| | Kinases | | Mouse |
| | (Plk1) | | |
| 12 | Aurora | Variety of different isoforms of kinases 1-3 and ABC | |
| | Kinases | | |
| 13 | Catanin | Antibodies to this protein with published epitope characteristics are | |
| | | not currently available from commercial sources. An antibody might | |
| | | be available by collaboration with an academic group, wherein the | |
| | | epitope of the antibody would be known, or could be discovered. | |
| | | Alternatively a new antibody could be created to an immunogenic | |
| | | sequence peculiar to this protein, and this sequence could be | |
| | | included in the calibration product | |
| 14 | Katanin | Synthetic peptide produced from human Katanin | Human |
| | | p80 aa 295-415 | |

### EXAMPLE 14

### Proteins Sharing a Common Location in the Cell

Lipid rafts is another physical location, which is a domain of the plasma membrane phase separated from surrounding regions of membrane. The phase separation arises as a consequence of the concentration of cholesterol and sphingomelin lipids, which group together to form a gel phase. Transmembrane proteins typically cannot enter this microdomain, which is populated instead by proteins anchored through fatty acid, or lipid-like units, including: GPI (glycosylinositolphosphatidyl) anchored proteins and proteins which are both myristolated and palmitoylated.

A series of proteins associated with lipid rafts include: (1) Lck (SRC kinase family members); (2) Fyn (SRC kinase family members); (3) H-Ras; (4) ZAP-70; (5) CD3ç; (6) LAT; (7) Flotillin-1; (8) CD2; (9) PAG; (10) F-actin; and (11) CD59. A calibration product comprising amino acid sequences to antibodies specific to multiple components above is conceivable. In some instances suitable antibodies with known epitope sequences have been described, in other instances such antibodies need to be identified. A product comprising the non-natural protein sequence comprising two or more epitopes selected from 1-11 as defined in Table 11 below would be useful in the study of lipid rafts in biology, biotechnology and medicine.

TABLE 11

| | | |
|---|---|---|
| Antibody for: | Epitope | Cross Reactivity |

| | | | |
|---|---|---|---|
| 1 | LcK | Synthetic peptide, corresponding to amino | Human |
| | | acids 22-36 of Human Lck p56 (CHYPIVPLD | |
| | | GKGTLL) (SEQ ID NO:59) | |
| 2 | Fyn | Peptie sequence VQCKDKEATKLTE (SEQ ID | Human |
| | | NO:60) corresponding to the C-terminus of | |
| | | FYN (NP_002028 | |
| 3 | H-Ras | SAKSVFSSKALVKMQLLKDVVGNDTYRINNKY | Human |
| | | DETYPPLPVEEIIKRSEFVIGQEVAYNLLVNNCE | |
| | | HFVTLLRYGEGVSEQANR (SEQ ID NO:61) | |
| 4 | ZAP-70 | Synthetic peptide KDKKLFLKRDNLLIAD | Human |
| | | (SEQ ID NO:62) derived from ZAP-70 | |
| | | sequence | |
| 5 | CD3epsilin | Synthetic peptide: KAKAKPVTRGAGA, (SEQ | Human, Baboon, |
| | | ID NO:63) corresponding to amino acids | Feline, Canine, |
| | | 156-168 of Human CD3 epsilon | Horse and Monkey |
| 6 | LAT | Synthetic peptide: GAPDYENLQELN, (SEQ | Human |
| | | ID NO:64) corresponding to amino acids | |
| | | 251-262 of Human LAT. | |
| 7 | Flotillin-1 | Synthetic peptide: SISQVNHKPLRTA, (SEQ | Human. |
| | | ID NO:65) corresponding to C terminal | Predicted to react |
| | | amino acids 415-427 of Human Flotillin 1. | with Chimpanzee |
| | | | and Pig due to |
| | | | sequence |
| | | | homology |

| | | |
|---|---|---|
| 8 | CD2 | Antibodies to this protein with published epitope characteristics |
| | | are not currently available from commercial sources. An antibody |
| | | might be available by collaboration with an academic group, |
| | | wherein the epitope of the antibody would be known, or could be |
| | | discovered. Alternatively a new antibody could be created to an |
| | | immunogenic sequence peculiar to this protein, and this |
| | | sequence could be included in the calibration product |

| | | | |
|---|---|---|---|
| 9 | PAG | Recombinant fragment, corresponding to amino | Human and |
| | | acids 97-432 of Human PAG (TLT CMQHYEEVQT | Mouse |
| | | SASDLLDSQD STGKPKCHQS RELPRIPPES | |
| | | AVDTMLTARS VDGDQGLGME GPYEVLKDSS | |
| | | SQENMVEDCL YETVKEIKEV AAAAHLEKGH | |
| | | SGKAKSTSAS KELPGPQTEG KAEFAEYASV | |
| | | DRNKKCRQSV NVESILGNSC DPEEEAPPPV | |
| | | PVKLLDENEN LQEKEGGEAE ESATDTTSET | |
| | | NKRFSSLSYK SREEDPTLTE EEISAMYSSV | |
| | | NKPGQLVNKS GQSLTVPEST YTSIQGDPQR | |
| | | SPSSCNDLYA TVKDFEKTPN STLPPAGRPS | |
| | | EEPEPDYEAI QTLNREEEKA TLGTNGHHGL | |
| | | VPKENDYESI SDLQQGRDIT RL) (SEQ ID NO:66) | |
| 10 | F-actin | Human CAPZA1, amino acids 80- | Human |
| | | 150(DLGNSRFLDPR NKISFKFDHL KEASDPQPE | |
| | | EADGGLKSWRESCDSALRAYVKDHYSNGFCTVYAKTI | |
| | | DGQ) (SEQ ID NO:67) | |

| | | |
|---|---|---|
| 11 | CD59 | Antibodies to this protein with published epitope characteristics |
| | | are not currently available from commercial sources. An antibody |
| | | might be available by collaboration with an academic group, |
| | | wherein the epitope of the antibody would be known, or could be |
| | | discovered. Alternatively a new antibody could be created to an |
| | | immunogenic sequence peculiar to this protein, and this |
| | | sequence could be included in the calibration product |

### EXAMPLE 15

### Macromolecular Complex

Macromolecular complexes which bring post-translational modification enzymes close to their target substrates are known. AKAPs (A-kinase anchoring proteins) are a good example of such complexes, which target enzymes involved in signalling with their substrate or effector proteins, creating local signalling circuits. The mAKAP macromoleular complex contains: (1) DE4D3; (2) Makap; (3) PKA; (4) Epac1; (5) Rap1; (6) MEKK; (7) MEK5; and (8) ERK5. A product comprising the non-natural protein sequence comprising two or more epitopes selected from 1-8 as defined in Table 12 below would be useful in the study of lipid rafts in biology, biotechnology and medicine.

### EXAMPLE 16

### Macromolecular Complex

Many biological polymers assemble stably or transiently into macromolecular complexes, which typically exhibit function. One such complex is the dystrophin complex which forms a junction between the plasma membrane of a muscle cell and the underlying cytoskeletal structure. The Dystrophin complex contains a number of proteins, including: (1) Laminin; (2) Alpha dystroglycan; (3) Beta dystroglycan; (4) Caveolin; (5) Dystrobrevin; (6) Dystrophin; (7) Actin; (8) Alpha sacroglycan; (9) Beta sacroglycan; (10) Delta sacroglycan; (11) Gamma sacroglycan; and (12) Sacrospan. A product comprising the non-natural protein sequence comprising two or more epitopes selected from 1-12 as defined in Table 13 below would be useful in the study of lipid rafts in biology, biotechnology and medicine.

TABLE 12

| | | |
|---|---|---|
| Antibody | | Cross |
| for: | Epitope | Reactivity |

| | | | |
|---|---|---|---|
| 1 | PDE4D3 | Synthetic peptide from unique region near N- | Human, |
| | | terminal of the PDE4D3 variant Antibodies to | Mouse and |
| | | this protein with published epitope | Rat |
| | | characteristics are not currently available | |
| | | from commercial sources. An antibody might | |
| | | be available by collaboration with an | |
| | | academic group, wherein the epitope of the | |
| | | antibody would be known, or could be | |
| | | discovered. Alternatively a new antibody | |
| | | could be created to an immunogenic | |
| | | sequence peculiar to this protein, and this | |
| | | sequence could be included in the calibration | |
| | | product | |
| 2 | mAKAP | Synthetic peptide LTMSVTLSPLRSQ-C, (SEQ | Human, |
| | | ID NO:68) representing the N-Terminus of | Mouse and |
| | | the human protein according to NP_004265 | Rat |
| 3 | PKA | KKGSEQESVKEFLAK (SEQ ID NO:69) (aa 8-22 | Human, |
| | | human alpha isoform of the catalytic subunit) | Bovine, |
| | | | Mouse, |
| | | | Porcine, Rat |
| 4 | Epac1 | Synthetic peptide: DHSVLTLQLPVTASVR, (SEQ | Human, |
| | | ID NO:70) corresponding to amino acids 526-541 | Mouse and |
| | | of Epac1 | Rat |
| 5 | Rap1 | Synthetic peptide: | Human |
| | | KAEEDPEAADSGEPQNKRTPDLPE, (SEQ ID | |
| | | NO:70) corresponding to amino acids 212-235 of | |
| | | Human RAP1 | |
| 6 | MEKK | Mouse Mekk-1 synthetic peptide (aa. 654-669; | Human, |
| | | Lange-Carter, 1993), conjugated to KLH | Mouse and |
| | | (LRAMLVYTPCHSLAER) (SEQ ID NO:71) | Rat |
| 7 | MEK5 | Synthetic peptide: AFEYEDEDGDRITVRSC (SEQ | Human, |
| | | ID NO:72), corresponding to amino acids 59/74 | Mouse, Rat, |
| | | of Human MEK5 | Rabbit, |
| | | | Xenopus |
| | | | laevis, Dog, |
| | | | Monkey and |
| | | | Sheep |
| 8 | ERK5 | Synthetic peptide: MAEPLKEEDG EDGS (SEQ ID | Human, |
| | | NO:73) conjugated to KLH, corresponding to N | Mouse, Rat |
| | | terminal amino acids 1-14 of Human ERK5 | and Cow |

TABLE 13

| | | |
|---|---|---|
| Antibody | | |
| for: | Epitope | Cross Reactivity |

| | | | |
|---|---|---|---|
| 1 | Laminin | Full length protein (Human) | Cross-reacts with all |
| | | | Mammals |
| 2 | alpha- | Antibodies to this protein with | Canine, Guinea Pig, |
| | dystroglycan | published epitope characteristics are | Human, Mouse, Rat, |
| | | not currently available from | Rabbit |
| | | commercial sources. An antibody | |
| | | might be available by collaboration | |
| | | with an academic group, wherein the | |
| | | epitope of the antibody would be | |
| | | known, or could be discovered. | |
| | | Alternatively a new antibody could | |
| | | be created to an immunogenic | |
| | | sequence peculiar to this protein, | |
| | | and this sequence could be included | |
| | | in the calibration product | |
| 3 | beta- | IVVEWT NNTLPLEPCP KEQIAGLSRR | Human |
| | dystroglycan | IAEDDGKPRP AFSNALEPDF | |
| | | KATSITVTGS GSCRHLQFIP | |
| | | VVPPRRVPSE APPTEVPDRD | |
| | | PEKSSEDDVY LHTVIPAVVV AAILLIA | |
| | | (SEQ ID NO:74) (from Human) | |
| 4 | Caveolin | M(1)SGGKYVDSEGHLYTVP(17)C | Human, Rat, Mouse, |
| | | (SEQ ID NO:75) (1-17 from human | Dog and Hamster |
| | | caveolin-1) | |
| 5 | dystrobrevin | ES IMGFRYRCQQ CHNYQLCQDC | Mouse, Rabbit, Rat |
| | | FWRGHAGGSH SNQHQMKEYT | |
| | | SWKSPAKKLT NALSKSLSCA | |
| | | SSREPLHPMF PDQPEKPLNL | |
| | | AHIVPPRPVT SMNDTLFSHS | |
| | | VPSSGSPFIT RSSPPKDSEV | |
| | | EQNKMLARAA PAFLKGRGIQ | |
| | | YSLNVADRLA DEH (SEQ ID NO:76) | |
| 6 | dystrophin | Fusion protein, corresponding to | Fish, Human, Mouse |
| | | amino acids 3200-3684 of Human | and Rat |
| | | Dystrophin. | |
| 7 | Actin | Synthetic peptide to the correct | |
| | | isoform of actin | |
| 8 | alpha- | Antibodies to this protein with | rabbit, mouse, |
| | sarcoglycan | published epitope characteristics are | human |
| | | not currently available from | |
| | | commercial sources. A new antibody | |
| | | could be created to an immunogenic | |
| | | sequence peculiar to this protein, | |
| | | and this sequence could be included | |
| | | in the calibration product | |
| 9 | beta- | MAAAAAAAAEQQSSNGPVKKSMREKA | Human |
| | sarcoglycan | VERRSVNKEHNSNFKAGYIPIDEDRLHK | |
| | | TGLRGRKGNLAICVIILLFILAVINLIITLV | |
| | | IWAVIRIGPNGCDSMEFHESGLLRFKQV | |
| | | SDMGVIHPLYKSTVGGRRNENLVITGN | |
| | | NQPIVFQQGTTKLSVENNKTSITSDIGM | |
| | | QFFDPRTQNILFSTDYETHEFHLPSGVK | |
| | | SLNVQKASTERITSNATSDLNIKVDGRA | |
| | | IVRGNEGVFIMGKTIEFHMGGNMELKAE | |
| | | NSIILNGSVMVSTTRLPSSSSGDQLGS | |
| | | GDWVRYKLCMCADGTLFKVQVTSQNM | |
| | | GCQISDNPCGNTH (SEQ ID NO:77) | |
| 10 | delta- | Synthetic peptide containing amino | Human. Does not |
| | sarcoglycan | acids 1-19 at the N terminus of | react with chicken, |
| | | human delta sacroglycan sequence | canine, hamster, |
| | | | mouse, rabbit, rat, |
| | | | pi |
| 11 | gamma- | Synethetic peptide containing amino | Human, chicken, |
| | sarcoglycan | acids 167-178 of rabbit gamma- | canine, mouse |
| | | sacroglycan sequence | |
| 12 | sarcospan | Antibodies to this protein with | |
| | | published epitope characteristics are | |
| | | not currently available from | |
| | | commercial sources. A new antibody | |
| | | could be created to an immunogenic | |
| | | sequence peculiar to this protein, | |
| | | and this sequence could be included | |
| | | in the calibration product | |

### EXAMPLE 17

### RyR2Macromolecular Complex Collection for Humans and Mice

The cardiac ryanodine receptor (RyR2), located in the sarcoplasmic reticulum (SR), is a calcium release channel which is centrally involved in the myocyte excitation-contraction (E-C). The ryanodine recpetor is also the center of a massive macromolecular complex, which includes numerous regulatory proteins that can modulate RyR2 function. This complex includes proteins that interact with the cytoplasmic part of the RyR2 directly or indirectly (e.g. calmodulin (CaM), FK-506-binding proteins, protein kinase A, Ca-CaM-dependent protein kinase, phosphatases 1 and 2A, mAKAP, spinophilin, PR130, sorcin, triadin, junctin, calsequestrin and Horner). Understanding both the physical/molecular nature of the protein-protein interactions between RyR and these other proteins is important since this complex and the modulation of the ryanodine receptor is believed to be involved in cardiac arrhythmias, pace-maker function of the heart and cardiac disease.

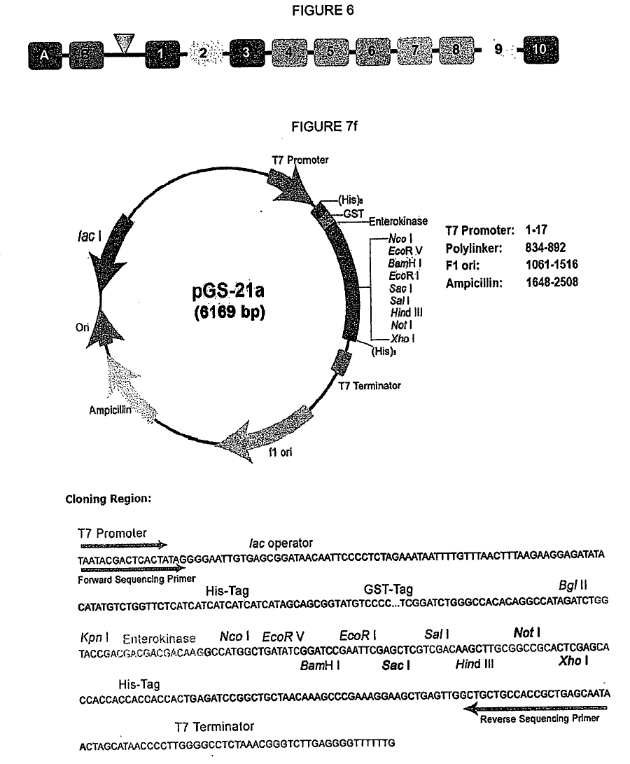

FIG. 5 shows all of the known protein-protein interaction sites between RyR2 and the macromolecular complex components. The proteins labelled with red numbers are some of the proteins which are involved in the complex, and which have been used to produce a corresponding calibrant. Antibody epitopes for the proteins in this complex have been genetically encoded and expressed in bacteria to produce a single protein (FIG. 6) that contains all of the antibody epitopes to each of the labelled proteins. The numerical notation of each protein corresponds to the notation in Table 14 below.

TABLE 14

| | | |
|---|---|---|
| Notation | Epitope | Amino Acid Sequence |

| | | |
|---|---|---|
| A | Histidine Tag | HHHHHH |
| | | (SEQ ID NO: 78) |
| B | Glutathione S | (whole protein) |
| | Transferase | |
| | (GST) | |
| 1 | Calmodulin | KRRWKKNFIAVSAANRFLLISSGAL |
| | Binding Peptide | (SEQ ID NO: 79) |
| | (CBP) | |
| 2 | RyR2 | DQQEQVKEDMETK |
| | | (SEQ ID NO: 80) |
| 3 | Calmodulin | DIDGDGQVNYEEFVQMMTAK |
| | | (SEQ ID NO: 81) |
| 4 | CamKII | CTRFTDEYQLFEEL |
| | | (SEQ ID NO: 82) |
| 5 | Calstabin 2 | GVQVETISPGDGR |
| | (FKBP12.6) | (SEQ ID NO: 83) |
| 6 | PKA | KKGSEQESVKEFLAK |
| | | (SEQ ID NO: 84) |
| 7 | PP1/PP2A | MSDSEKLNLDSIIGRLLE |
| | | (SEQ ID NO: 85) |
| 8 | Sorcin | MAYPGHPGAGGGYYPGGYGGAPGGP |
| | | AFPGQTQDPLYGYFAAVAGQDGQID |
| | | (SEQ ID NO: 86) |
| 9 | Calsequestrin | EGLNFPTYDGKDRVVSLSEKNFKQVL |
| | | KKYDLLCLYYHEPVSSDKVTQKQFQL |
| | | KEIVLELV |
| | | (SEQ ID NO: 87) |
| 10 | Histidine Tag | HHHHHH |
| | | (SEQ ID NO: 78) |

Notations A and B can be cleaved and removed, which allows for the production of the true RyR2 macromolecular complex epitope calibrant. Notations 1 and 10 are tags which can be used in purification of the calibrant protein and also act as common and widely used antibody epitopes.

(SEQ ID NO:88)

GGATCCAAACGCCGTTGGAAAAAGAACTTTATTGCAGTTAGTGCGGCCAA
TCGCTTTCTGCTGATTTCTTCGGGAGCGCTGGATCAGCAAGAACAGGTTA
AAGAAGATATGGAAACCAAAGATATCGATGGTGATGGGCAGGTGAACTAT
GAAGAATTCGTACAGATGATGACCGCCAAATGCACCCGTTTTACGGACGA
ATACCAGCTGTTCGAAGAACTGGGCGTTCAAGTTGAAACAATCAGCCCGG
GAGATGGCCGCAAAAAAGGTAGTGAGCAGGAATCTGTTAAAGAGTTCCTG
GCGAAAATGTCAGATTCGGAGAAACTGAATCTGGATAGCATTATTGGTCG
CCTGCTGGAGATGGCTTACCCGGGTCATCCGGGCGCGGGCGGCGGCTATT
ACCCAGGCGGCTATGGCGGTGCCCCGGGTGGTCCGGCGTTTCCGGGCCAG
ACCCAGGATCCGCTGTACGGCTATTTTGCAGCCGTGGCGGGCCAGGATGG
CCAAATCGATGAGGGCCTGAATTTCCCGACCTATGATGGGAAAGACCGTG
TTGTTTCACTGAGCGAAAAGAACTTTAAACAGGTACTGAAAAAATACGAT
CTGCTGTGCCTGTATTATCATGAACCAGTTTCCAGTGACAAAGTTACCCA
AAAACAGTTTCAGCTGAAGGAAATTGTACTGGAACTGGTGAAGCTT

The optimised genetic sequence, which encodes for all of the proteins in Table 14 is shown above as SEQ ID NO:88. Below as SEQ ID NO:89 is the resulting amino acid sequence from the genetic code

(SEQ ID NO:89)

KRRWKKNFIAVSAANRFLLISSGALDQQEQVKEDMETKDIDGDGQVNYEE
FVQMMTAKCTRFTDEYQLFEELGVQVETISPGDGRKKGSEQESVKEFLAK
MSDSEKLNLDSIIGRLLEMAYPGHPGAGGGYYPGGYGGAPGGPAFPGQTQ
DPLYGYFAAVAGQDGQIDEGLNFPTYDGKDRVVSLSEKNFKQVLKKYDLL
CLYYHEPVSSDKVTQKQFQLKEIVLELV.

The genetic sequence encoding the antibody epitopes that make up the calibrant (and purification tags) is synthesised and inserted into the E. coli expression vector pGS-21a (FIG. 7).

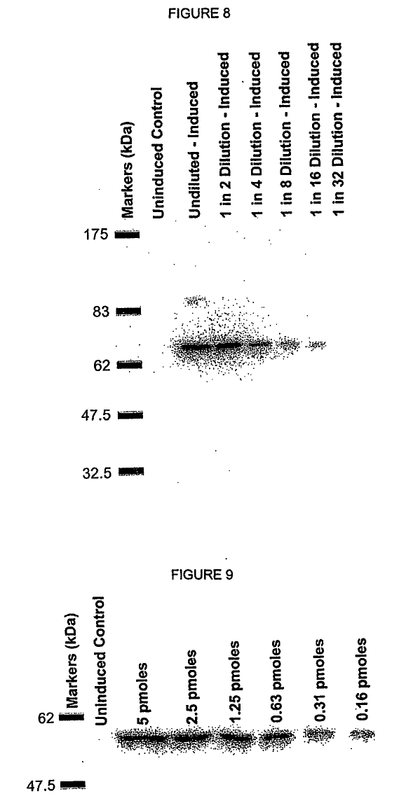

The pGS-21a plasmid, which now contains the genetic sequence to encode for the RyR2 macromolecular complex calibrant, is transformed into BL21 (DE3) pLysS E. coli cells. Transformed cells are selected and used to express the calibrant protein after the induction of gene synthesis with IPTG. After 3.5 hours of expression, the cells were harvested and re-suspended in sample buffer for analysis by SDS-PAGE and western blot. This was done in order to assess the purity of the calibrant product. FIG. 8 shows a western blot result of a doubling dilution series of the expressed calibrant.

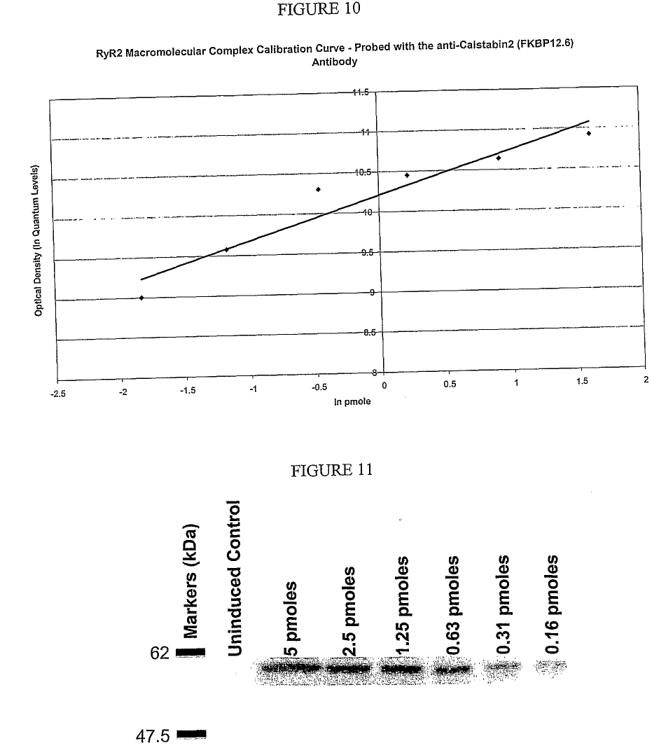

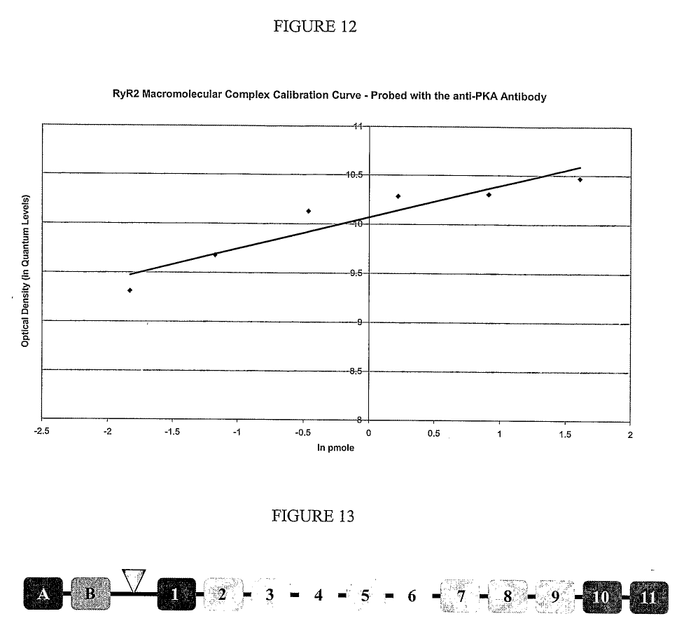

Following successful expression of a considerably pure calibrant product, the calibrant expression was scaled up from 1 ml to 1.5 L (FIGS. 9 and 11) and was used to produce a quantifiable calibration plot (FIGS. 10 and 12). The calibration curve was performed in triplicate and each replica was probed with a different epitope antibody and quantified by measuring the densitometry of the expressed RyR2Macromolecular Complex Calibrant.

### EXAMPLE 18

### MAPKinase Pathway Collection for Humans and Mice

Mitogen activated protein kinases (MAPK) are at the center of many signalling transduction pathways in eukaryotic cells. The study of MAPK pathways is important in the research of many disease areas such as inflammation, cancer and Parkinsons disease. We have used several proteins which are involved in one of the MAPK pathways to produce a corresponding calibrant. Antibody epitopes for the proteins on the in this pathway have been genetically encoded and expressed in bacteria to produce a single protein that contains all of the antibody epitopes to each of the labelled proteins. The numerical notation of each protein corresponds to the notation in Table 15 below.

TABLE 15

| | | |
|---|---|---|
| Notation | Epitope | Amino Acid Sequence |

| | | |
|---|---|---|
| A | Histidine Tag | HHHHHH |
| | | (SEQ ID NO:78) |
| B | Glutathione S | (whole protein) |
| | Transferase | |
| | (GST) | |
| 1 | Calmodulin | KRRWKKNFIAVSAANRFLLISSGAL |
| | Binding Peptide | (SEQ ID NO:90) |
| | (CBP) | |
| 2 | EGF Receptor | ETKPNGIFKGPTAENAEYLRVAPPSS |
| | | EFIGA |
| | | (SEQ ID NO:91) |
| 3 | Grb2 (Gab1) | KGACHGQTGMFPRNYVTPVNRNV |
| | | (SEQ ID NO:92) |
| 4 | SOS1 | KKSDHGNAFFPNSPSP |
| | | (SEQ ID NO:93) |
| 5 | Ras | EYDPTIEDSYRKQ |
| | | (SEQ ID NO:94) |
| 6 | A-Raf | QGPSPFTQQRDQEHFSF |
| | | (SEQ ID NO:95) |
| 7 | MEK1 | PKKKPTPIQLNP |
| | | (SEQ ID NO:96) |
| 8 | MEK2 | MLARRKPVLPALT |
| | | (SEQ ID NO:97) |
| 9 | ERK1 and ERK2 | PFTFDMELDDLPKERLKELIFQETAR |
| | | FQPGAPEAP |
| | | (SEQ ID NO:98) |
| 10 | RSK 1,2 and 3 | VSKMLHVDPHQRLTA |
| | | (SEQ ID NO:99) |
| 11 | Histidine Tag | HHHHHH |
| | | (SEQ ID NO:78) |

Notations A and B can be cleaved and removed, which allows for the production of the true MAPKinase pathway epitope calibrant. Notations 1 and 11 are tags which can be used in purification of the calibrant protein and also act as common and widely used antibody epitopes.

The optimised genetic sequence, which encodes for all of the proteins in Table 15 is shown below as SEQ ID NO:100.

(SEQ ID NO 100)

GGATCCAAACGTCGCTGGAAGAAAAATTTCATTGCGGTGTCGGCAGCGAA
TCGTTTTCTGCTGATCAGCTCTGGCGCTCTGGAGACAAAACCGAACGGTA
TCTTTAAAGGGCCAACAGCGGAAAATGCCGAATACCTGCGCGTGGCACCT
CCGTCTTCGGAGTTTATTGGCGCCAAGGGGGCATGCCACGGTCAGACCGG
TATGTTTCCTCGTAATTATGTGACCCCTGTTAACCGTAACGTCAAAAAAT
CCGATCACGGGAACGCATTCTTTCCGAATTCTCCGTCCCCGGAGTACGAT
CCGACTATTGAAGACAGTTACCGTAAACAGCAGGGACCTAGCCCGTTTAC
CCAACAGCGCGACCAGGAACATTTTAGTTTTCCGAAAAAGAAACCGACCC
CTATCCAGCTGAATCCGATGCTGGCTCGCCGCAAACCGGTCCTGCCAGCT
CTGACCCCTTTTACGTTCGATATGGAACTGGATGATCTGCCGAAGGAACG
CCTGAAGGAACTGATCTTTCAGGAAACCGCTCGTTTTCAGCCGGGTGCGC
CGGAAGCCCCGGTAAGCAAAATGCTGCACGTGGACCCGCATCAGCGCCTG
ACGGCAAAGCTT

The resulting amino acid sequence from the genetic sequence of SEQ ID NO:100 is given below as SEQ ID NO:101.

(SEQ ID NO: 101)

KRRWKKNFIAVSAANRFLLISSGALETKPNGIFKGPTAENAEYLRVAPPS
SEFIGAKGACHGQTGMFPRNYVTPVNRNVKKSDHGNAFFPNSPSPEYDPT
IEDSYRKQQGPSPFTQQRDQEHFSFPKKKPTPIQLNPMLARRKPVLPALT
PFTFDMELDDLPKERLKELIFQETARFQPGAPEAPVSKMLHVDPHQRL
TA.

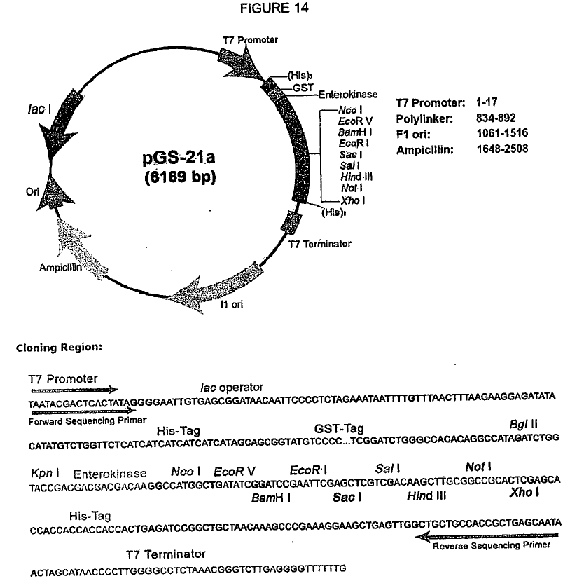

The genetic sequence encoding the antibody epitopes that make up the calibrant (and purification tags) is synthesised and inserted into the E. coli expression vector pGS-21a (FIG. 14).

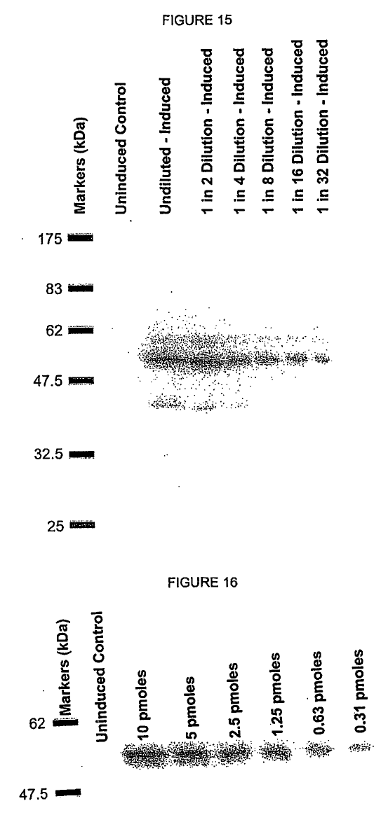

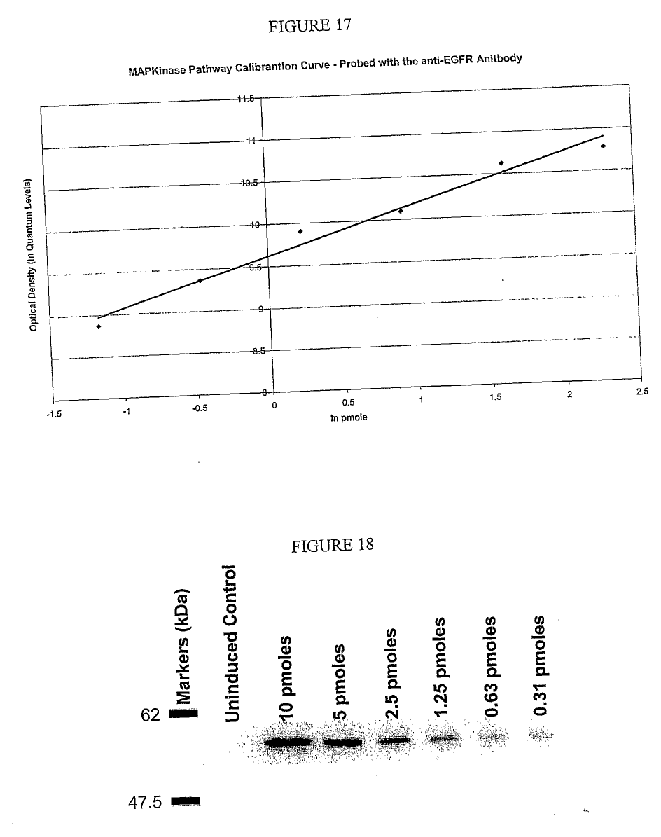

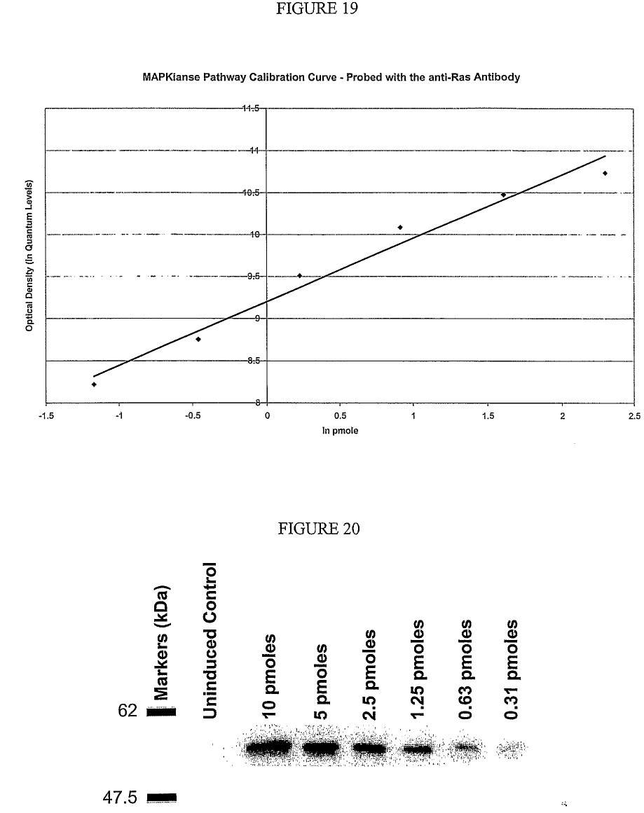

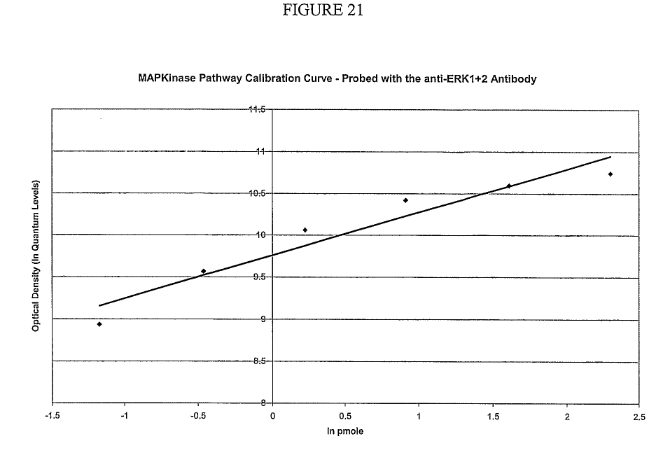

The pGS-21a plasmid, which now contains the genetic sequence to encode for the MAPK pathway calibrant, is transformed into BL21 (DE3) pLysS E. coli cells. Transformed cells are selected and used to express the calibrant protein after the induction of gene synthesis with IPTG. After 3.5 hours of expression, the cells were harvested and re-suspended in sample buffer for analysis by SDS-PAGE and western blot. This was done in order to assess the purity of the calibrant product. FIG. 15 shows a western blot result of a doubling dilution series of the expressed calibrant. Following successful expression of a considerably pure calibrant product, the calibrant expression was scaled up from 1 ml to 1.5 L (FIGS. 16, 18 and 20) and was used to produce a quantifiable calibration plots (FIGS. 17, 19 and 21). The calibration curve was performed in triplicate and each replica was probed with a different epitope antibody and quantified by measuring the densitometry of the expressed MAPKinase Pathway Calibrant.

### REFERENCES

1. Stamm S., Ben-Ari, S., Rafalska, I., Tang, Y., Zhang, Z., Toiber, D., Thanaraj, T. A., & Soreq, H. (2005) Function of alternative splicing. Gene 344, 1-20, 2005.)
2. Brandl C J., DeLoon S., Martin, D R., & MacLennan D H (1987) J. Biol. Chem. 262, 3768-74
3. Brandl C J., Green N M., Korczak B., & MacLennan D H (1986) Cell 44, 597-607
4. MacLennan D H., Brandl C J., Korczak B., & Green N M (1985) Nature 316, 696-700
5. Campbell A M., Kessler P D., & Fambrough D M. (1992) J. Biol. Chem. 267, 9321-25
6. Burk S E., Lytton J., MacLennan D H., & Shull G E. (1989) J. Biol. Chem. 264, 18561-8
7. Priori S G., Napolitano C., Memmi M., Colombi B., Drago F., Gasparini M., DeSimone L., Coltorti F., Bloise R., Keegan R., Cruz Filho F E S., Vignati G., Benatar A., & DeLogu A. (2002) Circulation 106, 69-74.

1. A non-natural protein sequence, the sequence comprising a removable scaffold portion and at least two protein or polypeptide epitopes, the protein or polypeptide epitopes being relationally linked.

2. The non-natural protein sequence of claim 1, wherein the relational link is selected from the group consisting of a common or shared functional relationship; isoforms of the same protein or polypeptide, or allelic variations thereof; and proteins or polypeptides that share a common location within a cell or that interact with the same target or protein or homologue thereof.

3. The non-natural protein sequence of claim 1, wherein the functional relationship is that the proteins or polypeptides are components of the same metabolic, catabolic or signal transduction pathway.

4. The non-natural protein sequence of claim 1 comprising a plurality of epitopes.

5. The non-natural protein sequence of claim 4 comprising more than 5 epitopes.

6. The non-natural protein sequence of claim 1, wherein the epitopes are:
(i) linked in series in a continuous length of sequence;
(ii) linked directly to one another;
(iii) attached to a backbone sequence comprising non-reactive or inert molecules;
(iv) linked so as to partially overlap;
(v) linked in series and interspersed with regions of an inert backbone sequence; or
(vi) linked or attached as a mixture of any of (i)-(iv) above.

7. The non-natural protein sequence of claim 1, wherein the epitopes are discontinuous in the non-natural protein sequence primary sequence, a number being present at the N-terminus, others being present at the C-terminus and optionally others being present within the sequence of the scaffold portion.

8. The non-natural protein of claim 1, wherein the epitopes are covalently attached to the scaffold portion at a site other than utilising the backbone sequence.

9. The non-natural protein sequence of claim 8, wherein the epitopes are attached using covalent bonding through unique or controlled numbers of reactive residues.

10. The non-natural protein sequence of claim 9, wherein the reactive residues are selected from the group consisting of cysteine, lysine, aspartate, glutamate, and tyrosine.

11. The non-natural protein sequence of claim 1, wherein the scaffold portion is non-reactive or innocuous and contributes to the calibration capability of the non-natural protein sequence in terms of its dominant physical properties.

12. The non-natural protein sequence f claim 1, wherein the scaffold portion and/or the epitope has a controlled property selected from the group consisting of its relative molecular mass (Mr) or weight (Mwt) and pH value for the isoelectric point of a given substance in solution (pI).

13. The non-natural protein sequence of claim 1, wherein the scaffold portion is a protein.

14. The non-natural protein sequence of claim 1, wherein the scaffold portion does not posses a chemically reactive group for attachment of the epitopes in series so the product is not branched.

15. The non-natural protein sequence claim 1, wherein the scaffold portion comprises at least one natural or unnatural amino acid with at least one or more chemically reactive groups.

16. The non-natural protein sequence of claim 15, wherein the one or more chemically reactive groups is within the side chain of a residue.

17. The non-natural protein of claim 15, wherein the one or more chemically reactive groups is selected from the group consisting of a carbonyl group on glutamic acid or aspartic acid, a hydroxyl group on tyrosine, at least one cysteine thiol group, and at least one lysine amino acid group.

18. The non-natural protein sequence of claim 17, wherein the number of chemically reactive groups comprising cysteine and/or lysine groups is controlled by selecting the scaffold portion from a natural source which comprises a desired number of reactive cysteine and/or lysine groups.

19. The non-natural protein sequence claim 15, wherein the scaffold portion is selected from the group consisting of: I27, from titin which comprises two cysteine residues; I39 domain which is a subunit (subunit 5) of splicing factor 3b and which comprises one cysteine residue, organ of Corti protein (Mus musculus) Swiss-Prot/TrEMBL Primary Accession Number Q8R448 which comprises one cysteine and one lysine residue; heat shock protein, mitochondrial (Mus musculus) Swiss-Prot/TrEMBL Primary Accession Number Q64433 which comprises eleven lysine residues; splicing factor 3B subunit 5 (Mus musculus) Swiss-Prot/TrEMBL Primary Accession Number Q923D4 which comprises one cysteine and five lysine residues; ubiquinol-cytochrome C reductase complex ubiquinone-binding protein QP-C (Schizosaccharomyces pomme) Swiss-Prot/TrEMBL Primary Accession Number P50523 which comprises one cysteine and six lysine residues; E1B protein (Human adenovirus type 11) Swiss-Prot/TrEMBL Primary Accession Number Q8B8U6 which comprises one cysteine residue; chaperonin (Arabidopsis thaliana) Swiss-Prot/TrEMBL Primary Accession Number P34893 which comprises nine lysine residues; photosystem II reaction centre H protein (Arabidopsis thaliana) Swiss-Prot/TrEMBL Primary Accession Number P56780 which comprises three lysine residues; a NADH-ubiquinone oxidoreductase subunit, mitochondrial [Precursor] (Homo sapiens) Swiss-Prot/TrEMBL Primary Accession Number P56181 which comprises one cysteine and nine lysine residues; signal recognition particle protein (Mus musculus) Swiss-Prot/TrEMBL Primary Accession Number P49962 which comprises two cysteine and eight lysine residues and DNA polymerase delta subunit 4(Mus musculus) Swiss-Prot/TrEMBL Primary Accession Number Q9CWP8 which comprises two cysteine and six lysine residues.

20. The non-natural protein sequence of claim 1, wherein the scaffold portion comprises one or more domains.

21. The non-natural protein sequence of claim 15, wherein the number of reactive cysteine and/or lysine groups is controlled by adding in or out or rendering ineffective any one or more of the reactive cysteine and/or lysine residues.

22. The non-natural protein sequence claim 15, wherein the scaffold portion is mutated to possess either one or no cysteine residues.

23. The non-natural protein sequence of claim 1, wherein the scaffold portion is 10 kDa.

24. The non-natural protein sequence of claim 1 comprising at least two independent epitopes relating to a single protein.

25. The non-natural protein sequence of claim 1, wherein the scaffold portion comprises a single reactive residue to allow covalent attachment of a second protein, peptide or hapten epitope comprising the covalent modification site, to which a modification-specific antibody exists.

26. Use of a non-natural protein sequence in detecting the presence of one or more relationally linked proteins or polypeptides in a sample and calibration of the said sample, the non-natural protein sequence product comprising a plurality of epitopes to proteins or polypeptides that are functionally related or linked and a scaffold portion comprising a non-reactive sequence that contributes to the calibration capability only in terms of its dominant physical properties.

27. Use of a non-natural protein sequence in detecting the presence of one or more relationally linked proteins or polypeptides and the absolute concentration of epitopes in a sample, the non-natural protein sequence product comprising a plurality of epitopes to proteins or polypeptides that are functionally related or linked and a scaffold portion comprising a non-reactive sequence that contributes to the calibration capability only in terms of its dominant physical properties.

28. Use according to claim 27 wherein the absolute concentration of the epitopes equates to a particular protein or protein modification.

29. Use of a non-natural protein sequence product comprising a plurality of epitopes to proteins or polypeptides that are functionally related or linked to calibrate a sandwich ELISA style experiment, wherein one antibody which is bound to a physical surface captures an epitope of the non-natural protein sequence and a second antibody, specific for a second feature on the same epitope binds to the captured eptiope.

30. (canceled)

31. A kit for identifying the presence of and quantifying the amount of, at least two relationally linked proteins or polypeptides in a sample, the kit comprising a non-natural protein sequence product comprising a scaffold portion comprising a non-reactive sequence that contributes to the calibration capability only in terms of its dominant physical properties and plurality of epitopes to proteins or polypeptides that are functionally related or linked.

32. A method of simultaneously detecting the presence of at least two relationally linked proteins or polypeptides and quantifying the amount of said functionally related or linked proteins or polypeptides in a sample, the method comprising:
a) providing a non-natural protein sequence, the sequence comprising a scaffold portion and at least two protein or polypeptide epitopes, the proteins or polypeptides being relationally linked and mixing this non-natural protein sequence with a test sample that may or may not contain binding partners to the epitopes;
b) adding at least two antibodies specific for the at least two epitopes;
c) carrying out a separation detection technique on said non-natural protein sequence; and optionally
d) generating at least one comparison point comprising intensity of a signal produced by the scaffold portion of the non-natural protein sequence.

33. (canceled)
